(19) United States
(12) Patent Application Publication
(10) Pub. No.: US 2024/0409864 A1
(43) Pub. Date: Dec. 12, 2024

(54) Apparatus and Methods for Generating and Analyzing Three-Dimensional Cellular Materials

(71) Applicant: Agilent Technologies, Inc., Santa Clara, CA (US)

(72) Inventors: Chong Wing Yung, Santa Clara, CA (US); Smruti Madan Phadnis, Santa Clara, CA (US); Andrew C. Neilson, Santa Clara, CA (US); Hien Vuong Cheung, Santa Clara, CA (US)

(21) Appl. No.: 18/701,642

(22) PCT Filed: Nov. 4, 2022
(86) PCT No.: PCT/US2022/048957

§ 371 Date: Apr. 16, 2024

Related U.S. Application Data
(60) Provisional application No. 63/276,099, filed on Nov. 5, 2021.
(60) Provisional application No. 63/320,912, filed on Mar. 17, 2022.

(51) Int. Cl. C12M 1/32; C12M 1/00; C12M 1/34; C12M 3/00

(52) U.S. Cl. CPC C12M 23/12; C12M 21/08; C12M 23/22; C12M 23/34; C12M 41/00

(57) ABSTRACT

An apparatus for containing a three-dimensional cellular material surrounded by a medium that facilitates and maintains the centering of the three-dimensional cellular material throughout an assay is provided. The apparatus includes a well having an open proximal end and a closed distal end. Further, the well defines a compartment having an interior surface and a sample nesting site for containing the three-dimensional cellular material surrounded by the medium. A central indentation is located at the closed distal end of the well, a first concentric lip is located above the central indentation in a y-direction towards the open proximal end of the well, and a second concentric lip is located above the first concentric lip in the y-direction towards the open proximal end of the well. Additionally, the first concentric lip and the second concentric lip define a groove therebetween. A method of forming a three-dimensional cellular material is also provided.

### RELATED APPLICATIONS

The present application claims priority to U.S. Provisional Application Ser. No. 63/276,099, filed on Nov. 5, 2021, and 63/320,912, filed on Mar. 17, 2022, which are incorporated herein in their entirety by reference thereto.

### BACKGROUND

In vitro three-dimensional culture systems, though more complex than two-dimensional cell culture systems, have proven to have several advantages that can mimic in vivo cellular behavior. Examples of three-dimensional culture systems include, but are not limited to spheroids, organoids, and embryoids. Spheroids are three-dimensional cellular materials made from a single cell type, such as, for example, established cell lines. Organoids are three-dimensional cellular materials made from multiple cell type to, for example, closely resemble a target organ. Embryoids are three-dimensional cellular materials made from pluripotent stem cells and include the representative cells from the three germ layers. Cells grown in these scaffold-free platforms generate and organize their own three-dimensional extracellular matrix, where cell-cell interactions dominate over cell-substrate interaction, without added materials, and the three-dimensional structures closely resemble in vivo tissues.

The emergence of ultra-low attachment (ULA) plates has enabled spheroids and other three-dimensional cellular materials to offer an easy route to access to three-dimensional cell culture models due to ease of generation and scalability to high throughputs. ULA plates can generate uniform spheroids and other three-dimensional structures in terms of size, shape, and time needed to form a spheroid. This consistency has enabled the adoption of such three-dimensional structures for various applications such as in disease modelling, drug discovery and safety, tissue engineering, and regenerative medicine.

Currently, spheroid microplates for analytical analysis such as via a Seahorse XFe96 instrument are commercially available. Such plates allow metabolic assessment of single spheroids and micro tissues. However, there are limitations to the existing plate design. First, spheroids cannot be generated or cultured in a universal plate that is then used for metabolic analysis using the Seahorse instrument. Instead, spheroids must first be generated in a culture plate, such as an ultra-low attachment plate or a hanging droplet plate, before being manually transferred to another spheroid plate for metabolic analysis. This manual transfer process is quite laborious and time consuming and is also prone to failure due to loss or damage of the small, delicate spheroids. Further, of the fraction of spheroids that are successfully transferred from the culture plate to the metabolic analysis plate, many of the spheroids do not stay centered during the mixing cycles that must be completed to carry out the metabolic analysis. This lack of centering can lead to erratic metabolic measurements or even a complete loss of signal when the spheroid is completely displaced from the measurement region of a well in the culture plate. Further, a lack of centering can also lead to challenges while imaging the spheroids as well, as many commercially available plates have a broad base that results in the spheroids moving out of the focal plane. Additionally, current spheroid microplates have a large microchamber volume, which is a temporary volume of liquid that is formed when the sensor cartridge is lowered into the wells of the microplate to carry out the metabolic analysis. This large microchamber volume reduces the sensitivity of the metabolic assay and also necessitates the use of larger spheroids having a diameter of about 500 micrometers to generate a measurable signal, while spheroids or other three-dimensional cellular materials having diameters or about 100 micrometers to about 250 micrometers are preferred. Further, currently available spheroid microplates are not amenable to more sophisticated applications such as organ-on-chip based metabolic assays or co-culture based metabolic assays.

As such, what is needed is a microplate and a method of forming spheroids and other three-dimensional cellular materials that addresses the problems discussed above with current microplates for forming, culturing, centering, and assaying three-dimensional cellular materials.

### SUMMARY

In one embodiment, the present disclosure is directed to an apparatus for containing a three-dimensional cellular material surrounded by a medium. The apparatus includes a well having an open proximal end and a closed distal end that defines a bottom of the well. Further, the well defines a compartment having an interior surface and a sample nesting site for containing the three-dimensional cellular material surrounded by the medium. Additionally, a central indentation is located at the closed distal end of the well, a first concentric lip is located above the central indentation in a y-direction towards the open proximal end of the well, and a second concentric lip is located above the first concentric lip in the y-direction towards the open proximal end of the well. In addition, the first concentric lip and the second concentric lip define a groove therebetween.

In one embodiment, the first concentric lip can have a first concave radius of curvature, the second concentric lip can have a second concave radius of curvature, and the central indentation can have a third concave radius of curvature.

In another embodiment, the interior surface of the compartment near the closed distal end of the well can be defined by a first convex radius of curvature between the central indentation and the first concentric lip and a second convex radius of curvature between the first concentric lip and the second concentric lip.

In still another embodiment, the bottom of the well can be transparent.

In yet another embodiment, a coating can be deposited on at least a portion of the interior surface of the compartment, and the coating can facilitate the collection of the three-dimensional cellular material at the central indentation at the closed distal end of the well.

In one more embodiment, at least one protrusion can be located at the closed distal end of the well, and the at least one protrusion can be spaced radially from the central indentation towards a sidewall of the well.

In one embodiment, the interior surface can include polyethylene terephthalate, polystyrene, polypropylene, polyvinyl chloride, cyclic olefin copolymer, polycarbonate, or a combination thereof.

In another embodiment, the apparatus can include a probe that forms a seal at the closed distal end of the well when introduced into the compartment, and the first concentric lip or the second concentric lip defines a planar surface for receiving the probe. Further, the probe can include a sensor for measuring a parameter, such as a metabolic parameter, and the well can hold a volume of medium beneath the seal that is less than 200 microliters. For instance, the volume can range from about 0.25 microliters to about 1.75 microliters.

In still another embodiment, the three-dimensional cellular material can include a spheroid, an organoid, or a tissue sample.

In yet another embodiment, the apparatus can include a plurality of wells defining a plurality of compartments.

In another particular embodiment, the present disclosure is directed to a method for forming a three-dimensional cellular material. The method includes the steps of: a) providing a plate having at least one well having an open proximal end and a closed distal end that defines a bottom of the well, wherein the well defines a compartment having an interior surface and a sample nesting site for containing the three-dimensional cellular material, wherein a central indentation is located at the closed distal end of the well, a first concentric lip is located above the central indentation in a y-direction towards the open proximal end of the well, and a second concentric lip is located above the first concentric lip in the y-direction towards the open proximal end of the well, wherein the first concentric lip and the second concentric lip define a groove therebetween; b) adding cells and a medium to the compartment; and c) allowing the three-dimensional cellular material to form from the cells.

In one embodiment, the first concentric lip can have a first concave radius of curvature, and the second concentric lip can have a second concave radius of curvature, and the central indentation can have a third concave radius of curvature.

In another embodiment, the interior surface of the compartment near the closed distal end of the well can be defined by a first convex radius of curvature between the central indentation and the first concentric lip and a second convex radius of curvature between the first concentric lip and the second concentric lip.

In still another embodiment, the bottom of the well can be transparent.

In yet another embodiment, a coating can be deposited on at least a portion of the interior surface of the compartment, and the coating can decrease a level of attachment of the three-dimensional cellular material to the interior surface.

In one more embodiment, at least one protrusion can be located at the closed distal end of the well, and the at least one protrusion can be spaced radially from the central indentation towards a sidewall of the well.

In an additional embodiment, the interior surface can include polyethylene terephthalate, polystyrene, polypropylene, polyvinyl chloride, polycarbonate, cyclic olefin copolymer, or a combination thereof.

In another embodiment, the method can further include the step of measuring a parameter of the three-dimensional cellular material by introducing a probe into the compartment to form a seal near the closed distal end of the well, wherein the first concentric lip or the second concentric lip defines a planar surface for receiving the probe. Further, the probe can include a sensor for measuring the parameter, such as a metabolic parameter. In addition, a volume of medium contained within the well beneath the seal can be less than 200 microliters. For instance, the volume of medium can range from about 0.25 microliters to about 1.75 microliters.

In still another embodiment, the three-dimensional cellular material can include a spheroid, an organoid or a tissue sample.

In yet another embodiment, the three-dimensional cellular material can have a radius in a x-direction and a radius in a y-direction, wherein a ratio of the radius in the x-direction to the radius in the y-direction ranges from about 0.75 to about 1.25 after step (c).

In one more embodiment, the plate can include a plurality of wells defining a plurality of compartments.

Other features and aspects of the present disclosure are discussed in greater detail below.

### BRIEF DESCRIPTION OF THE DRAWINGS

A full and enabling disclosure of the present disclosure is set forth more particularly in the remainder of the specification, including reference to the accompanying figures, in which:

FIG. 1 is a top view of one embodiment of a cell culture apparatus contemplated by the present disclosure;

FIG. 2 is a cross-sectional view of a portion of the cell culture apparatus shown in FIG. 1 taken at dashed line C-C and also includes a zoomed in view of a portion of the cross-sectional view of FIG. 2 illustrating the geometry of a well contemplated by the present disclosure;

FIG. 3 is another cross-sectional view of a well of a cell culture apparatus contemplated by the present disclosure;

FIG. 4 is a perspective view of the well of FIG. 3;

FIG. 5 is bottom view of the well of FIG. 3;

FIG. 6 is a cross-sectional view of the well of FIG. 5 taken at dashed line 6-6;

FIG. 7 is a top view of one embodiment of a cell culture apparatus contemplated by the present disclosure;

FIGS. 8 and 9 are upright and inverted, respectively, exploded perspective view of a cell culture apparatus (e.g., a multi-well plate) and a covered cartridge adapted to mate with the plate showing various features of the cell culture apparatus according to one embodiment of the present disclosure;

FIG. 10 is a schematic illustration of a measurement system and apparatus in accordance with one embodiment of the present disclosure;

FIG. 11 is a top view photograph of spheroids cultured in a different apparatus and then transferred to a well of the apparatus contemplated by the present disclosure versus those cultured in the apparatus contemplated by the present disclosure;

FIG. 12 is a schematic of an example of three-dimensional cellular material formed according to the methods contemplated by the present disclosure;

FIG. 13 is a graph showing the oxygen consumption rate (OCR) versus time measurements for spheroids cultured in a different apparatus and transferred to the apparatus contemplated by the present disclosure compared to spheroids cultured directly in the apparatus contemplated by the present disclosure;

FIG. 14 is a top view photograph of spheroids cultured in the apparatus contemplated by the present disclosure where each well in the apparatus was polished and included 20 microliters of coating material, where the left column shows the spheroids before the OCR assay was conducted and the right column shows the spheroids after the OCR assay was conducted;

FIG. 15 is a graph showing the OCR versus time for the spheroids shown in FIG. 14;

FIG. 16 is a top view photograph of HepG2 spheroids cultured in the apparatus contemplated by the present disclosure where each well in the apparatus was unpolished and included 50 microliters of a coating material, where the left column shows the spheroids before the OCR assay was conducted and the right column shows the spheroids after the OCR assay was conducted;

FIG. 17 is a graph showing the OCR versus time for the spheroids shown in FIG. 16;

FIG. 18 is a top view photograph of HepG2 spheroids cultured in the apparatus contemplated by the present disclosure, where each well in the apparatus was polished and included 50 microliters of coating material, where the left column shows the spheroids before the OCR assay was conducted and the right column shows the spheroids after the OCR assay was conducted;

FIG. 19 is a graph showing the OCR versus time for the spheroids shown in FIG. 18;

FIG. 20 is a top view photograph of a HepG2 spheroid cultured in the apparatus contemplated by the present disclosure, where the spheroid was generated using centrifugation;

FIG. 21 is a top view photograph of a Panc1 spheroid cultured in the apparatus contemplated by the present disclosure, where the spheroid was generated using centrifugation;

FIG. 22 is a top view photograph of a C2C12 spheroid cultured in the apparatus contemplated by the present disclosure, where the spheroid was generated using centrifugation;

FIGS. 23A, 23B, and 23C are top view photographs of a HepG2 spheroid cultured in the apparatus contemplated by the present disclosure, where the apparatus was coated with a 2-methacryloyloxyethyl phosphorylcholine polymer or MPC polymer (e.g., Lipidure®), followed by centrifugation;

FIGS. 24A, 24B, and 24C are top view photographs of a HepG2 spheroid cultured in the apparatus contemplated by the present disclosure, where the apparatus was coated with BioFLOAT®, followed by centrifugation;

FIG. 25 is a graph showing the oxygen consumption rate (OCR) of spheroids formed from HepG2 cells cultured in the apparatus contemplated by the present disclosure with a BioFLOAT® coating, followed by centrifugation;

FIG. 26 is a graph showing the extra cellular acidification rate (ECAR) of a spheroid formed from HepG2 cells cultured in the apparatus contemplated by the present disclosure with a BioFLOAT® coating, followed by centrifugation;

FIG. 27 is a series of top view photographs of various HepG2 spheroids after the OCR and ECAR assays summarized in FIGS. 25 and 26 were completed;

FIG. 28A is a top view photograph of a Panc1 spheroid cultured in the apparatus contemplated by the present disclosure, where the apparatus was coated with BioFLOAT®, followed by centrifugation, where the spheroid has a diameter of about 425 micrometers;

FIG. 28B is a top view photograph of a Panc1 spheroid cultured in the apparatus contemplated by the present disclosure, where the apparatus was coated with BioFLOAT®, followed by centrifugation, where the spheroid has a diameter of about 550 micrometers;

FIG. 28C is a top view photograph of a Panc1 spheroid cultured in the apparatus contemplated by the present disclosure, where the apparatus was coated with BioFLOAT®, followed by centrifugation, where the spheroid has a diameter of about 750 micrometers;

FIG. 29 is a graph showing the oxygen consumption rate (OCR) of a spheroid formed from Panc1 cells cultured in the apparatus contemplated by the present disclosure with a BioFLOAT® coating, followed by centrifugation, where the spheroid has a diameter of about 425 micrometers;

FIG. 30 is a graph showing the extra cellular acidification rate (ECAR) of spheroids formed from Panc1 cells cultured in the apparatus contemplated by the present disclosure with a BioFLOAT® coating, followed by centrifugation, where the spheroid has a diameter of about 425 micrometers;

FIG. 31 is a graph showing the oxygen consumption rate (OCR) of a spheroid formed from Panc1 cells cultured in the apparatus contemplated by the present disclosure with a BioFLOAT® coating, followed by centrifugation, where the spheroid has a diameter of about 750 micrometers;

FIG. 32 is a graph showing the extra cellular acidification rate (ECAR) of spheroids formed from Panc1 cells cultured in the apparatus contemplated by the present disclosure with a BioFLOAT® coating, followed by centrifugation, where the spheroid has a diameter of about 750 micrometers;

FIG. 33A is a top view photograph of a HepG2 spheroid cultured in the apparatus contemplated by the present disclosure, where the apparatus was coated with BioFLOAT®, followed by centrifugation, where the spheroid has a diameter of about 370 micrometers;

FIG. 33B is a top view photograph of a HepG2 spheroid cultured in the apparatus contemplated by the present disclosure, where the apparatus was coated with BioFLOAT®, followed by centrifugation, where the spheroid has a diameter of about 450 micrometers;

FIG. 33C is a top view photograph of a HepG2 spheroid cultured in the apparatus contemplated by the present disclosure, where the apparatus was coated with BioFLOAT®, followed by centrifugation, where the spheroid has a diameter of about 525 micrometers;

FIG. 34 is a graph showing the oxygen consumption rate (OCR) of a spheroid formed from HepG2 cells cultured in the apparatus contemplated by the present disclosure with a BioFLOAT® coating, followed by centrifugation, where the spheroid has a diameter of about 370 micrometers;

FIG. 35 is a graph showing the extra cellular acidification rate (ECAR) of a spheroid formed from HepG2 cells cultured in the apparatus contemplated by the present disclosure with a BioFLOAT® coating, followed by centrifugation, where the spheroid has a diameter of about 370 micrometers;

FIG. 36 is a graph showing the oxygen consumption rate (OCR) of a spheroid formed from HepG2 cells cultured in the apparatus contemplated by the present disclosure with a BioFLOAT® coating, followed by centrifugation, where the spheroid has a diameter of about 525 micrometers;

FIG. 37 is a graph showing the extra cellular acidification rate (ECAR) of a spheroid formed from HepG2 cells cultured in the apparatus contemplated by the present disclosure with a BioFLOAT® coating, followed by centrifugation, where the spheroid has a diameter of about 525 micrometers;

FIG. 38 is a series of top view photographs of various C2C12 spheroids cultured in the apparatus contemplated by the present disclosure, where the apparatus was coated with BioFLOAT®, where the spheroids have a diameter of about 150 micrometers;

FIG. 39 is a top view photograph of a C2C12 spheroid cultured in the apparatus contemplated by the present disclosure, where the apparatus was coated with BioFLOAT®, without centrifugation;

FIG. 40 is a top view photograph of a Panc1 spheroid cultured in the apparatus contemplated by the present disclosure, where the apparatus was coated with BioFLOAT®, without centrifugation;

FIG. 41 is a bar graph illustrating that ATP levels are proportional to spheroid size, where the apparatus used to form the spheroids was coated with BioFLOAT®;

FIG. 42 is a graph showing the oxygen consumption rate (OCR) of spheroids formed from Panc1 cells cultured in the apparatus contemplated by the present disclosure with a BioFLOAT® coating, without centrifugation, where the spheroids had diameters of about 325 micrometers and about 250 micrometers;

FIG. 43 is a graph showing the extra cellular acidification rate (ECAR) of spheroids formed from Panc1 cells cultured in the apparatus contemplated by the present disclosure with a BioFLOAT® coating, without centrifugation, where the spheroids had diameters of about 325 micrometers and about 250 micrometers;

FIG. 44 is a graph showing the oxygen consumption rate (OCR) of spheroids formed from C2C12 cells cultured in the apparatus contemplated by the present disclosure with a BioFLOAT® coating, without centrifugation, where the spheroids had diameters of about 180 micrometers and about 140 micrometers;

FIG. 45 is a graph showing the extra cellular acidification rate (ECAR) of spheroids formed from C2C12 cells cultured in the apparatus contemplated by the present disclosure with a BioFLOAT® coating, without centrifugation, where the spheroids had diameters of about 180 micrometers and about 140 micrometers;

FIG. 46 is an image of the 140 micrometer spheroids assayed to determine the OCR and ECAR of FIGS. 44 and 45;

FIG. 47 is an image of the 180 micrometer spheroids assayed to determine the OCR and ECAR of FIGS. 44 and 45;

FIG. 48 is an image of the 250 micrometer spheroids assayed to determine the OCR and ECAR of FIGS. 42 and 43;

FIG. 49 is an image of the 325 micrometer spheroids assayed to determine the OCR and ECAR of FIGS. 42 and 43;

FIG. 50 is a graph showing the oxygen consumption rate (OCR) of spheroids formed from Panc1 cells cultured in the apparatus contemplated by the present disclosure with a BioFLOAT® coating, where the spheroids had diameters of about 400 micrometers;

FIG. 51 is an image of the 400 micrometer spheroids assayed to determine the OCR of FIG. 50;

FIG. 52 is a graph showing the oxygen consumption rate (OCR) of spheroids formed from C2C12 cells cultured in the apparatus contemplated by the present disclosure with a BioFLOAT® coating, where the spheroids had diameters of about 180 micrometers;

FIG. 53 is an image of the 180 micrometer spheroids assayed to determine the OCR of FIG. 52;

FIG. 54 is a confocal image of a spheroid formed from Panc1 cells cultured in the apparatus contemplated by the present disclosure with a BioFLOAT® coating, where the spheroid had a diameter of about 350 micrometers;

FIG. 55 is a confocal image of a spheroid formed from Panc1 cells cultured in the apparatus contemplated by the present disclosure with a BioFLOAT® coating, where the spheroid had a diameter of about 500 micrometers;

FIG. 56 is a confocal image of a spheroid formed from C2C12 cells cultured in the apparatus contemplated by the present disclosure with a BioFLOAT® coating, where the spheroid had a diameter of about 100 micrometers;

FIG. 57 is a confocal image of a spheroid formed from C2C12 cells cultured in the apparatus contemplated by the present disclosure with a BioFLOAT® coating, where the spheroid had a diameter of about 150 micrometers;

FIG. 58 is a confocal image of a spheroid formed from C2C12 cells cultured in the apparatus contemplated by the present disclosure with a BioFLOAT® coating and stained with Cell Tracker Orange;

FIG. 59 is a confocal image of a spheroid formed from C2C12 cells cultured in the apparatus contemplated by the present disclosure with a BioFLOAT® coating and stained with Calcein AM;

FIG. 60 is a confocal image of a spheroid formed from HepG2 cells cultured in the apparatus contemplated by the present disclosure with a BioFLOAT® coating and stained with Hoescht 34580;

FIG. 61 is a graph showing the basal oxygen consumption rate (OCR) of liver spheroids acquired and then transferred for culture in the apparatus contemplated by the present disclosure;

FIG. 62 is an image of the spheroid with the lowest OCR from FIG. 63, showing many loose cells around the perimeter of the spheroid, indicating that the spheroid may have been damaged during transfer;

FIG. 63 is a graph showing the oxygen consumption rate (OCR) of liver spheroids cultured in the apparatus contemplated by the present disclosure, where the plate was made of polystyrene and the cartridge for the Mito Stress Test was made out of polycarbonate;

FIG. 64 is a graph showing the oxygen consumption rate (OCR) of liver spheroids cultured in the apparatus contemplated by the present disclosure, where the plate and the cartridge for the Mito Stress Test were made of polyethylene terephthalate (PET);

FIG. 65 is a graph showing the oxygen consumption rate (OCR) of PANC1 spheroids cultured and assayed in the apparatus contemplated by the present disclosure;

FIG. 66 is a graph showing the oxygen consumption rate (OCR) during a Mito Stress Test of PANC1 spheroids cultured on a commercial plate and then transferred to the apparatus contemplated by the present disclosure for testing, without centrifugation; and

FIG. 67 is a graph showing the oxygen consumption rate (OCR) during a Mito Stress Test of PANC1 spheroids cultured on a commercial plate and then transferred to the apparatus contemplated by the present disclosure for testing, with centrifugation.

For the sake of clarity, data presented may be further improved through implementation of geometry specific algorithm refinement.

Repeat use of reference characters in the present specification and drawings is intended to represent the same or analogous features or elements of the present invention.

### DETAILED DESCRIPTION

It is to be understood by one of ordinary skill in the art that the present discussion is a description of exemplary embodiments only and is not intended as limiting the broader aspects of the present disclosure.

Example aspects of the present disclosure are directed to an apparatus and methods for analyzing one or more metabolic parameters associated with three-dimensional materials (e.g., a spheroid, an organoid, etc.). The apparatus and methods of the present disclosure allow for the controlled growth and subsequent metabolic analysis of such three-dimensional materials in a single well plate without requiring a separate plate for growth and analysis, thus eliminating the material transfer step required by many conventional systems.

The apparatus of the present disclosure can be incorporated into any suitable system designed to analyze three dimensional materials. For example, the apparatus of the present disclosure can be incorporated into all types of cell metabolic analysis systems, microfluidic systems, microplate readers, multimode and absorbance readers, and imaging systems.

One particular device that has made great advances is the SEAHORSE analysis platform that is manufactured and sold by Agilent Technologies. The SEAHORSE analysis platform, for instance, can make quantitative measurements of mitochondrial function and cellular bioenergetics. For example, the instrument can measure oxygen concentration and pH in the extracellular media of a cell-based assay. Different aspects of the SEAHORSE analysis platform are described in U.S. Pat. Nos. 7,276,351, 7,638,321, 8,697,431, 9,170,253, U.S. Patent Publication No. 2014/0170671, U.S. Patent Publication No. 2015/0343439, U.S. Patent Publication No. 2016/0077083, and U.S. Patent Publication No. 2016/0096173, which are all incorporated herein by reference. The apparatus and methods of the present disclosure can be incorporated into the above-described devices for providing various advantages and benefits.

The system and process of the present disclosure can also be incorporated into microplate readers including multimode and absorbance readers. For example, the detection system of the present disclosure can be incorporated into various exemplary devices including the SYNERGY Hybrid Multimode Readers, the CYTATION Hybrid Multimode Reader, the LOGPHASE Microbiology Readers, the EPOCH Microplate Spectrophotometers, the ELx808 Absorbance Reader, and the 800 TS Absorbance Reader all available through Agilent Technologies.

Generally speaking, the present disclosure is directed to is directed to an apparatus for containing a three-dimensional cellular material surrounded by a medium. The apparatus includes a well having an open proximal end and a closed distal end that defines a bottom of the well. Further, the well defines a compartment having an interior surface and a sample nesting site for containing the three-dimensional cellular material surrounded by the medium. Additionally, a central indentation is located at the closed distal end of the well, a first concentric lip is located above the central indentation in a y-direction towards the open proximal end of the well, and a second concentric lip is located above the first concentric lip in the y-direction towards the open proximal end of the well. In addition, the first concentric lip and the second concentric lip define a groove therebetween. The present disclosure is also directed to a method for forming a three-dimensional cellular material. The method includes the steps of: a) providing a plate having at least one well having an open proximal end and a closed distal end that defines a bottom of the well, wherein the well defines a compartment having an interior surface and a sample nesting site for containing the three-dimensional cellular material, wherein a central indentation is located at the closed distal end of the well, a first concentric lip is located above the central indentation in a y-direction towards the open proximal end of the well, and a second concentric lip is located above the first concentric lip in the y-direction towards the open proximal end of the well, wherein the first concentric lip and the second concentric lip define a groove therebetween; b) adding cells and a medium to the compartment; and c) allowing the three-dimensional cellular material to form from the cells.

Such an apparatus and method allow for the in-situ generation of three-dimensional cellular materials such as spheroids and organoids and subsequent assaying of the three-dimensional cellular material in the same plate, thus eliminating the inefficient transferring step that can also lead to inaccurate assay results. Furthermore, in the event a transfer is needed, the transfer is made more efficient and accurate using a multi-channel pipette or by automation due to the specific geometry of the bottom of the well, which also facilitates centering of the three-dimensional cellular material in the cell culture apparatus. Further, the geometry uses gravity to drive in situ generation and self-centering of the three-dimensional cellular material during the performance of an assay and during mixing cycles and imaging as well. In addition, the microchamber volume formed during the measurement cycles is about half of the volume of standard commercially available well plates, which improves accuracy of any measurements taken due to the high signal to background ratio. This also allows for smaller three-dimensional cellular materials to be analyzed efficiently. Moreover, the well plate is specifically designed to engage a sensor probe to measure one or more parameters, such as metabolic parameters, and contains a planar surface on which the probe sits, which eliminates the need for complicated inserts and the related cumbersome processes involved with the use of such inserts.

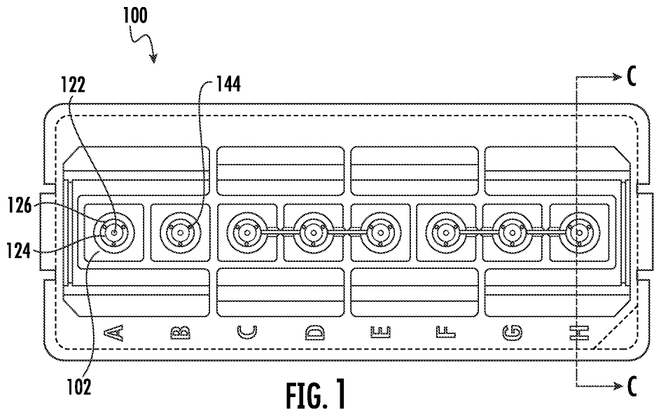

Turning now to FIG. 1, one embodiment of a cell culture apparatus 100 contemplated by the present disclosure is shown. Although the cell culture apparatus 100 is shown with eight wells 102, it is to be understood that a cell culture apparatus having any number of wells 102 (e.g., 1, 4, 6, 8, 12, 16, 24, 48, 96, 384, 1536 or any other number of wells) is contemplated by the present disclosure. Regardless of the number of wells 102 contained in the cell culture apparatus 100, each well 102 can include a central indentation 122, a first concentric lip 124, and a second concentric lip 126 positioned outside the first concentric lip 124. In addition, each well 102 can include one or more peripheral protrusions 144 as shown.

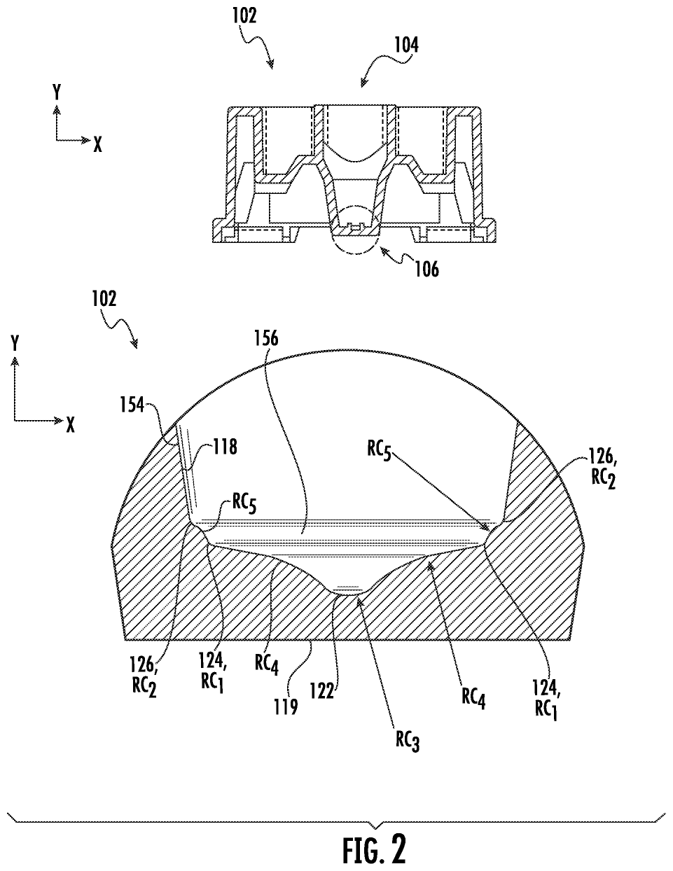

FIG. 2 is a cross-sectional view of a portion of the cell culture apparatus 100 shown in FIG. 1 taken at dashed line C-C and also includes a zoomed in view of a portion of the cross-sectional view of FIG. 2 illustrating the geometry of a well 102 contemplated by the present disclosure. The well 102 has an open proximal end 104 and a closed distal end 106 in the y-direction, with a sidewall 154 extending therebetween to define an interior surface 118 of the well 102. In addition, the central indentation 122 is located at the closed distal end 106 of the well 102 at the bottom 119 of the well 102, the first concentric lip 124 is located above the central indentation 122 in the y-direction towards the open proximal end 104 of the well 102, and the second concentric lip 126 is located above the first concentric lip 124 in the y-direction towards the open proximal end 104 of the well 102. Further, the first concentric lip 124 and the second concentric lip 126 define a groove 156 therebetween. As shown, the first concentric lip 124 can have RC₁ a first concave radius of curvature RC₁, while the second concentric lip 126 can have a second concave radius of curvature RC₂, and the central indentation 122 can have a third concave radius of curvature RC₃. Meanwhile, the interior surface 118 of the well 102 near the closed distal end 106 can be defined by a first convex radius of curvature RC₄ that is located between the central indentation 122 and the first concentric lip 124, as well as a second convex radius of curvature RC₅ that is located between the first concentric lip 124 and the second concentric lip 126.

The first concave radius of curvature RC₁ can range from about 25 micrometers to about 200 micrometers, such as from about 50 micrometers to about 175 micrometers, such as from about 75 micrometers to about 100 micrometers, or any ranges therebetween. Further, the second concave radius of curvature RC₂ can also range from about 50 micrometers to about 175 micrometers, such as from about 75 micrometers to about 100 micrometers, or any ranges therebetween. In addition, the third concave radius of curvature RC₃ can range from about 400 micrometers to about 600 micrometers, such as from about 425 micrometers to about 575 micrometers, such as from about 450 micrometers to about 550 micrometers, or any ranges therebetween. Moreover, the first convex radius of curvature RC₄ can range from about 0.75 millimeters to about 2.25 millimeters, such as from about 1 millimeter to about 2 millimeters, such as from about 1.25 millimeters to about 1.75 millimeters, or any ranges therebetween. Lastly, the second convex radius of curvature RC₅ can range from about 175 micrometers to about 325 micrometers, such as from about 200 micrometers to about 300 micrometers, such as from about 225 micrometers to about 275 micrometers, or any ranges therebetween. Without intending to be limited by any particular theory, the present inventors have found that such a configuration for the well 102 creates an environment amenable to the formation of three-dimensional cellular materials that are centered with a more compact geometry that resembles in vivo tissue growth more accurately compared to three-dimensional cellular materials formed by currently available cell culture plates, which tend to exhibit a pancake-like geometry, while also allowing for a reduced volume of media for more accurate assay measurements and increased sensitivity.

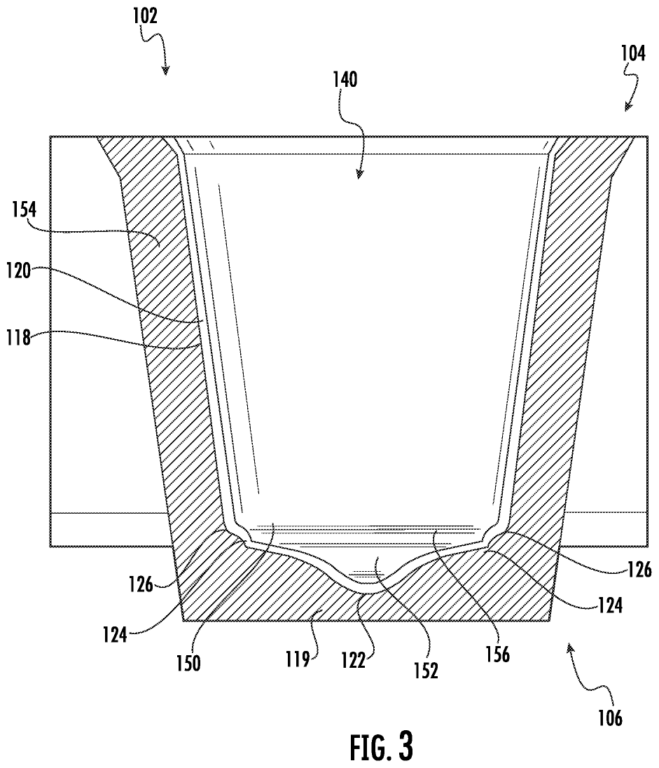

Turning now to FIG. 3, each well 102 can include a coating 120 on the interior surface 118 of the well 102. Specifically, the coating 120 can be deposited on at least a portion of the interior surface 118, where it has been found that the coating 120 can increase the smoothness of the interior surface 118 and can decrease the level of attachment of any three-dimensional cellular material to the interior surface 118. In some embodiments, the well 102 can be formed from a molded polymer that can include a polyethylene terephthalate, a polystyrene, a polypropylene, a polyvinyl chloride, a polycarbonate, a cyclic olefin copolymer, or a combination thereof. The coating 120 can be suitable coating agent including but not limited to polymer coating agents. For instance, the polymer coating agent can be a poloxamer (e.g., Pluronic® F-127 or Pluronic® P-188) or a 2-methacryloyloxyethyl phosphorylcholine polymer or MPC polymer (e.g., Lipidure® from AMSBIO). Other examples of suitable coating agents include RinseAid from STEMCELL Technologies and BioFLOAT® from faCellitate. In addition, it should be understood that the bottom 119 of the well 102 can be transparent to enable imaging or other photometric measurements. Further, in some embodiments, the interior surface 118 of the well 102 can be polished to enhance the transparency of the well 102.

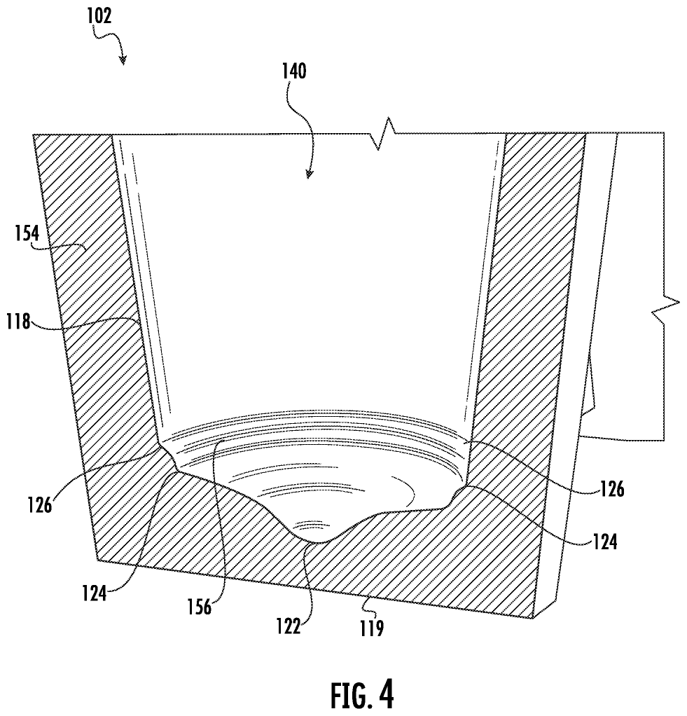

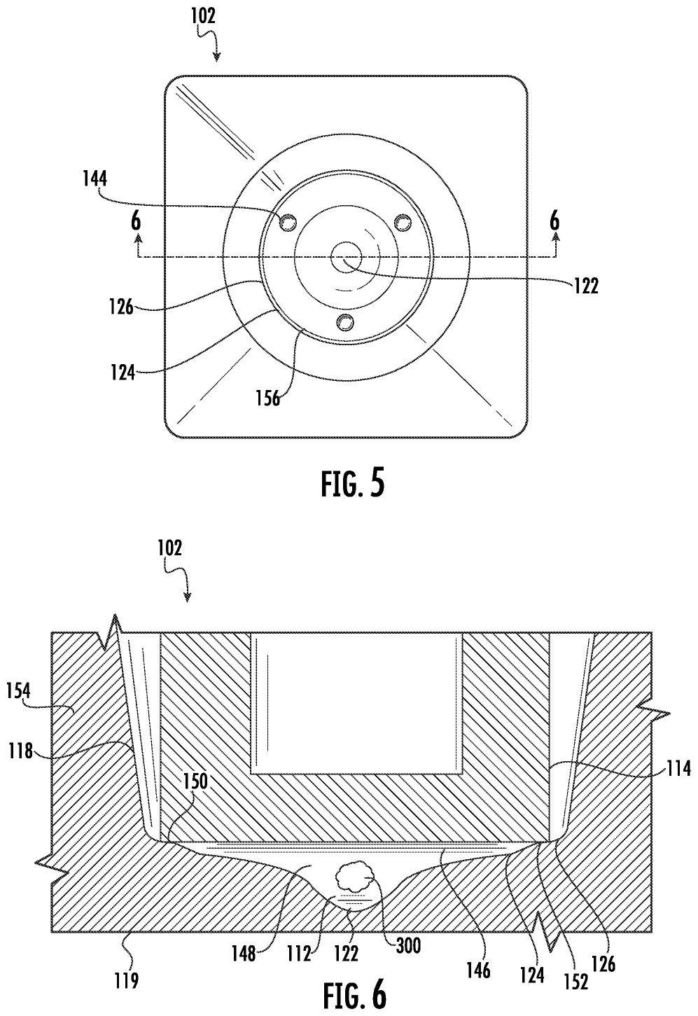

Turning now to FIG. 4, a perspective view of the well 102 of FIG. 3 is illustrated, while FIG. 5 is bottom view of the well 102 of FIG. 3. As shown, the well 102 includes a compartment 140, and the central indentation 122, the first concentric lip 124 and the second concentric lip 126 are illustrated in perspective detail, where the first concentric lip 124 and the second concentric lip 126 define a groove 156 therebetween. Further, as shown in FIG. 5, the well 102 can include one or more peripheral protrusions 144 located at the closed distal end 106 of the well 106, which can be spaced radially from the central indentation 122 towards a sidewall 154 (see FIG. 6) of the well 102. The present inventors have found that the central indentation 122 can restrict the movement of a spheroid or other three-dimensional cellular material 300 in the well 102 (see FIG. 6) to a limited space or area that corresponds, for instance, with the focal plane of any imager such that the spheroid or other three-dimensional cellular material 300 remains centered, while the protrusions 144 can prevent a testing cartridge from damaging any coating present, such as an ultra-low attachment coating.

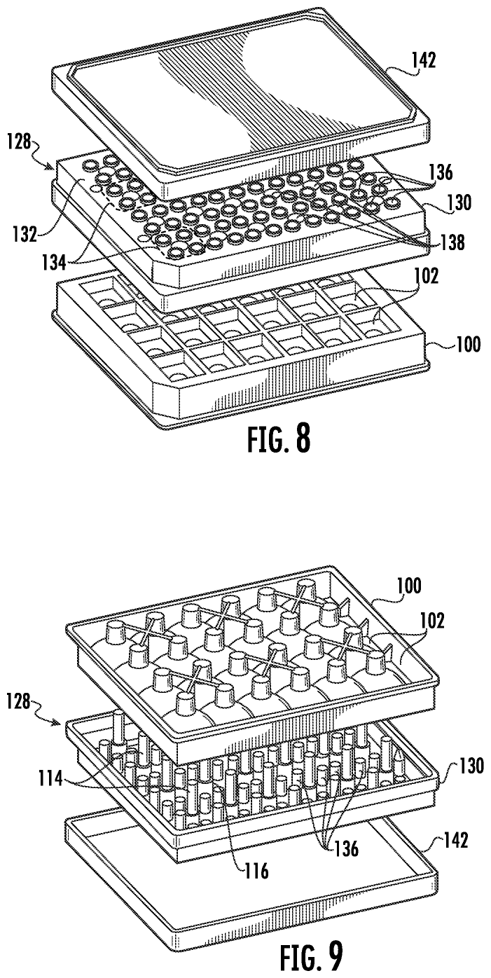

FIG. 6 is a cross-sectional view of the well 102 of FIG. 5 taken at dashed line 6-6, where three-dimensional cellular material 300 is contained within a sample nesting site 112 formed by the central indentation 122 within the compartment 140 and is surrounded by a medium 148 (e.g., cell culture medium). Further, when a probe 114 is inserted into the well 102, the space defined by the probe 114 and the central indentation 122 creates a microchamber 146 for containing the three-dimensional cellular material 300 within medium 148 for analysis of a parameter. Based on the geometric configuration of the well 102 of the present disclosure, the present inventors have discovered that the volume of the microchamber 146 can be reduced significantly to provide for more accurate parameter measurements. For instance, the volume of the microchamber 146 can be less than 200 microliters, such as less than 100 microliters, such as less than 50 microliters, such as less than 25 microliters, such as less than 10 microliters, such as less than 5 microliters, such as less than 2 microliters. For example, the volume can range from about 0.25 microliters to about 1.75 microliters, such as from about 0.3 microliters to about 1.5 microliters, such as from about 0.4 microliters to about 1.4 microliters, or any ranges therebetween. Although any parameter can be measured by methods described in more detail with respect to FIGS. 8-10 below, in some embodiments, the parameter is a metabolic parameter such as oxygen consumption rate (OCR), extra cellular acidification rate (ECAR), and pH. As shown, the probe 114 can form a seal 152 at the closed distal end 106 of the well 102 when introduced into the compartment 140, and the first concentric lip 124 or the second concentric lip 126 can create a planar surface 150 for receiving the probe 114 depending on the size of the probe 114 utilized.

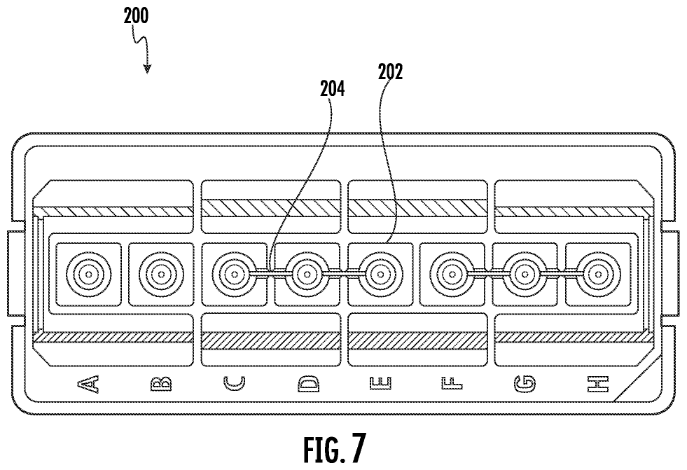

It should also be understood, as shown in FIG. 7, that the present disclosure contemplates a cell culture apparatus 200 that can be in the form of a well plate similar to the apparatus 100 shown in FIG. 1 but that includes wells 202 that are interconnected with a channel 204 between adjacent wells. Such a configuration allows for an organ-on-chip application to be utilized in conjunction with the methods contemplated by the present disclosure and allows for simultaneous metabolic analysis of multi-organoid-based organ-on-chip co-culture models.

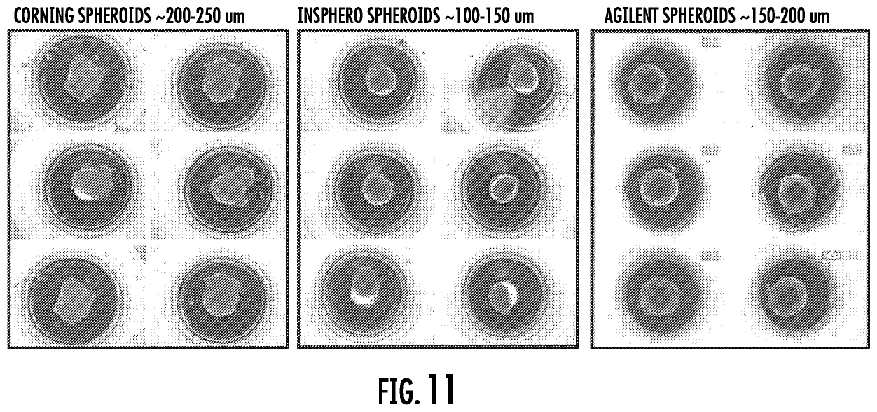

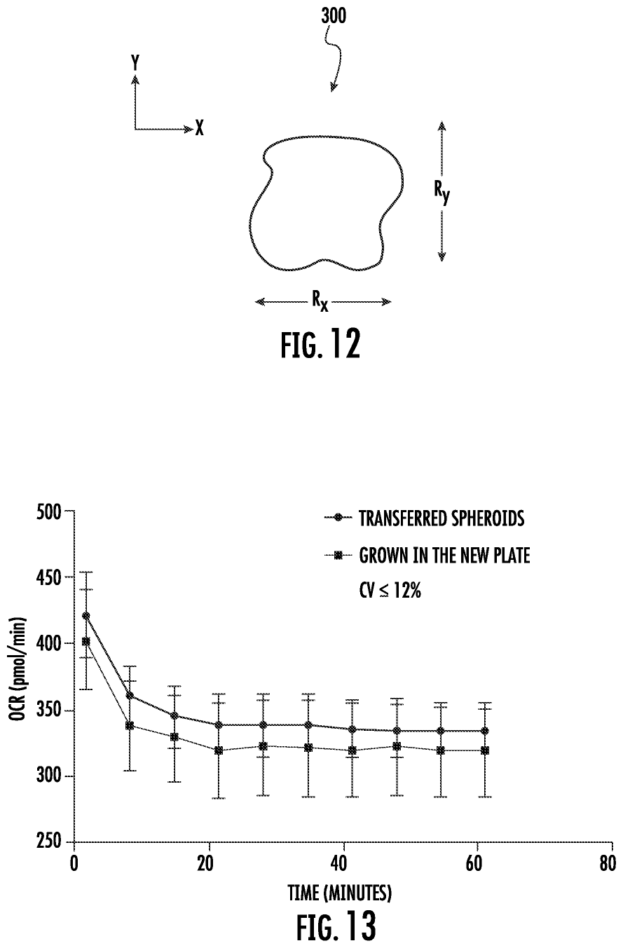

In any event, and referring the FIGS. 11 and 12, the three-dimensional cellular material 300 formed by the apparatus 100, 200 or any other apparatus contemplated by the present disclosure (the right set of images in FIG. 11) can be compact and of a uniform size and shape in terms of the radius of the three-dimensional cellular material 300 in the x-direction, Rx, and y-direction, Rc, compared to the more pancake-like, non-uniform geometries of the material cultured in other cell culture apparatuses (the left two sets of images in FIG. 11). For instance, the ratio of the radius in the x-direction, Rx, to the radius in the y-direction, Ry, can range from about 0.75 to about 1.25, such as from about 0.8 to about 1.2, such as from about 0.85 to about 1.15, such as from about 0.9 to about 1.1, or any ranges therebetween. Further, the three-dimensional cellular material 300 can have a radius (or diameter) Rx or Ry ranging from about 25 micrometers to about 500 micrometers, such as from about 30 micrometers to about 400 micrometers, such as from about 40 micrometers to about 300 micrometers, such as from about 50 micrometers to about 250 micrometers, or any ranges therebetween.

FIGS. 8 and 9 are upright and inverted, respectively, exploded perspective view of a cell culture apparatus (e.g., a multi-well plate) and a covered cartridge adapted to mate with the plate showing various features of the cell culture apparatus according to one embodiment of the present disclosure;

Referring to FIGS. 8 and 9, a well plate configuration suitable for carrying out the parameter testing mentioned above and practicing embodiments of the disclosure is shown. It includes a cell culture apparatus 100 defining a plurality of wells 102. The cell culture apparatus 100 can be combined with a cartridge 128 and removable cover 142. In the depicted embodiment, the cell culture apparatus 100 has 24 wells, but it should be understood that the number of wells 102 in a plate may vary from 1 to several thousand. In some embodiments, a single well of nearly any size may be fabricated, or multiple wells may be fabricated, or multiple wells may be fabricated in a one- or two-dimensional arrangement. In various embodiments, one may exploit a two-dimensional pattern of wells corresponding to the pattern and dimensions of a microplate as described for example by the Society for Biomolecular Screening standards for microplates (“SBS-1 Footprints” and “SBS-4 Well Positions,” both full proposed standards updated May 20, 2003). The plates can include 1, 4, 6, 8, 12, 16, 24, 48, 96, 384, 1536, or any other number of individual wells. The larger numbers of wells present engineering challenges because of the fine structure required to practice embodiments of the present disclosure. The cartridge 128 is a generally planar element comprising a frame 130 made, e.g., from molded plastics. The planar surface 132 defines a plurality of regions 134 that correspond to, or register with, a number of the respective openings of a plurality of wells 102 defined in the cell culture apparatus 100. Within each of these regions 134, in the depicted embodiment, the planar element defines first, second, third, and fourth ports 136, which serve as reservoirs for delivery of gases or reagents, and a central aperture 138 to a probe 114 containing one or more sensors 116. Each of the ports is adapted to hold and to release on demand a test fluid to the respective well 102 beneath it. The ports 136 are sized and positioned so that groups of four ports may be positioned over the wells 102, and a gas or test fluid from any one of the four ports may be delivered to a respective well 102. In some embodiments, the number of ports in each region may be less than four or greater than four. The ports 136 and probes 114 may be compliantly mounted relative to the cell culture apparatus 100 so as to permit it to nest within the microplate by accommodating lateral movement. The construction of the microplate to include compliant regions permits its manufacture to looser tolerances and permits the cartridge to be used with slightly differently dimensioned microplates. Compliance can be achieved, for example, by using an elastomeric polymer to form planar surface 132, so as to permit relative movement between frame 130 and the probes 114 and ports 136 in each region.

Each of the ports 136 may have a cylindrical, conical, or cubic shape, open through planar surface 132 at the top, and closed at the bottom except for a small hole, i.e., a capillary aperture, typically centered within the bottom surface. The capillary aperture is adapted to retain test fluid in the port, e.g., by surface tension, absent an external force, such as a positive pressure differential force, a negative pressure differential force, or possibly a centrifugal force. Each port may be fabricated from a polymer material that is impervious to gasses, test compounds, or from any other solid material. When configured for use with a cell culture apparatus 100, the liquid volume contained by each port may range less than 200 microliters, such as less than 100 microliters, such as less than 50 microliters, such as less than 25 microliters, such as less than 10 microliters, such as less than 5 microliters, such as less than 2 microliters. For example, the volume can range from about 0.25 microliters to about 1.75 microliters, such as from about 0.3 microliters to about 1.5 microliters, such as from about 0.4 microliters to about 1.4 microliters, or any ranges therebetween.

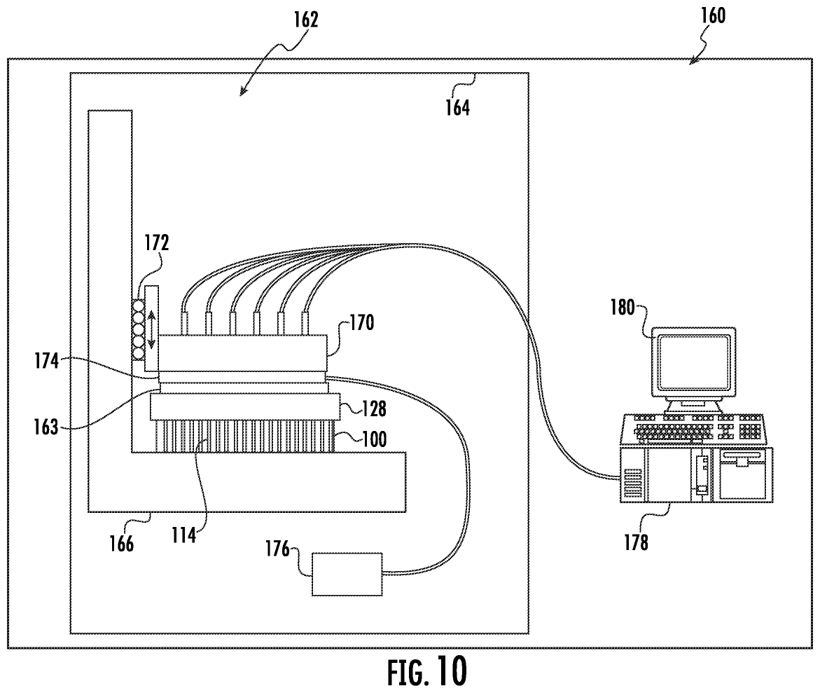

FIG. 10 shows a schematic of a measurement system and apparatus (e.g., an analyzer) used in connection with embodiments of the present disclosure. It comprises an analyzer 160 including a compound storage and delivery apparatus 162 disposed in a housing 164 (shown in dashed lines) and includes a cartridge 128 defining a plurality of apertures for receiving sensor structures and a plurality of fluid ports (shown in detail in FIGS. 8 and 9) compliantly mounted, and a stage or base 166 adapted to receive a cell culture apparatus 100, e.g., a cell culture plate. The cartridge 128 is disposed above, and adapted to mate with, the cell culture apparatus 100. The cartridge 128 optionally is held by a cartridge holder 168 adapted to receive the cartridge 128. The apparatus also includes a mounting block 170, which can reciprocate as shown by the double headed arrow, preferably powered by a motor (not shown), including an elevator mechanism 172. The elevator mechanism 172 may be adapted to move the cartridge 128 relative to the stage 166, or the cell culture apparatus 100. The mounting block 170 includes a gas multiplexer 174 attached to a gas supply or gas reservoir 176. The gas supply 176 is in fluid communication with the cartridge 128 and is used to impel the delivery of test fluid from a port in the cartridge 128 to a well 102 in the cell culture apparatus 100, or to fix the gas composition in one or more wells 102. A plurality of probes 114 with sensors 116 are adapted for insertion into the plurality of apertures in the cartridge 128 and may be used to gather data indicative of the state of cells disposed in wells in the cell culture apparatus 100.

The compound storage and delivery apparatus 162 is controlled by a controller 178, that may be integrated with a computer 180, that may control the elevator mechanism 172, the multiplexer 174, and the gas supply or reservoir 176. The controller 178 may, thereby, permit delivery of a test fluid from a port 136 to a corresponding well 102 when an associated sensor 116 is disposed in the well 102.

The apparatus described herein is a modification of the apparatus disclosed in US Patent Application Publication No. 2008/0014571, the disclosure of which is incorporated herein by reference, and enables experimentation with and analysis of three-dimensional cell culture samples, such as a tissue sample, a biopsied sample, or a cell scaffold holding cells. Viability of the sample may be maintained and control exercised over its microenvironment. In certain embodiments, a gas may be added to the media or to a headspace in the well above the media to modify the microenvironment about the sample by altering dissolved gas composition. In certain other embodiments, a solution of a biologically active substance may be added to the media to modify the microenvironment about the sample by exposing the sample to a biologically active substance. A metered amount of one or more gases and/or one or more drugs or other solutes may be added to media in the well to set the microenvironment in the medium about the sample to a predetermined point. The microenvironment in the well may be set to a hypoxic condition. The concentration of one or more solutes in media about the sample may be measured. A plurality of measurements, separated in time, of the concentration of one or more solutes in media about the sample may be taken.

A common test performed on the system described above is a mitochondrial stress test. In this assay a series of injections are delivered through the drug ports of the cartridge in order to measure the response of the biological sample to various compounds (oligomycin, FCCP, rotenone and antimycin). These compounds are preloaded into a drug reservoir (port) on the XF cartridge prior to execution of the assay. When the cartridge is inserted into the instrument it is coupled to a manifold which when activated by a solenoid valve, provides pneumatic pressure to the head space of the reservoir forcing the compound through a small orifice and into the well containing the biological sample. The pneumatic manifold and valve system may be modified to redirect one of these ports to an external gas supply (gas cylinder or bottle). The gas supply may be connected to the instrument through a port on the rear connector panel. The bottle may be located near the instrument and may contain a regulator and bubbler for humidification of the incoming gas. When activated, a solenoid valve may open, allowing the gas to flow through the manifold/cartridge interface, through the drug port orifice, and into the head space above the biological sample. By oscillating the probe vertically, the gas will be mixed with the medium allowing control of the available oxygen to the sample. For example, by perfusing argon into the head space, the available oxygen in the medium is displaced and a more hypoxic condition is created around the sample. By turning off the gas and mixing, ambient levels of oxygen may be re-established.

In some embodiments, a source of a solution of a biologically active substance may be in fluid communication with media in wells for exposing a sample to the substance.

To control the operation and timing of the solenoid valve, the instrument software may be modified to facilitate control of the valve/timing and to expose some of the calculation variables used during calibration. For example, to calculate molar concentration of oxygen in the medium, the concentration at calibration is preferably known and input into the calculation table. Under some conditions the initial calibration value (F or current ambient concentration) may not be known. In this case, calibration may be achieved by injecting sodium sulfite into a set of control wells and calibrating the system based on a known value. To calculate these results, certain coefficients may be made accessible in the software as would be understood by those of ordinary skill in the art. A separate window may be created in the software to facilitate access to these variables, valve control and calculation of calibration coefficients.

The instrument may be tested using a well characterized cell line to verify proper operation and control of the gas system. A series of tests may be conducted to demonstrate the ability to purge oxygen from medium and create a hypoxic microenvironment around the sample. These tests may include: 1. Calibration of the instrument under known and unknown ambient O2 concentration; and 2. Verify performance of the gas delivery system and the ability to drive environmental oxygen levels to desired value (<5% PPO). This may be verified within the instrument by looking at the oxygen level data. The readout from the instrument may provide a view that presents this data.

An alternative to controlling oxygen and pH within the sample environment may be to enclose the entire instrument in an environmental chamber and pump down the chamber to the desired levels. This alternative approach may be less desirable, as it may be very costly, take up a lot of lab space, and require long periods of time to achieve the desired levels around the tissue. By the time these oxygen levels are achieved the tissue may be dead.

### EXAMPLES

The following examples illustrate certain exemplary and preferred embodiments and applications of the present disclosure but are not intended to be illustrative of all embodiments and applications.

### Example 1

First, Panc1/HepG2 cells were grown in the well plates generally contemplated by the present disclosure but with only a single concentric lip as well as commercially available plates, followed by the transfer of the spheroids cultured in the commercially available plates to the plates of the present disclosure. The experimental protocol was as follows:
1. Add 50 ul of Lipidure solution (prepared in ETOH) to the plates. Leave overnight at 37 degrees C. or at 50 degrees C. for 1 hour for the ETOH to evaporate.
2. Trypsinize the HepG2 with multiple washes of Mg—Ca free PBS to make single cells. Leave in incubator at 37 deg C. for 2-3 mins.
3. Transfer cells to centrifugal tube and add PBS to fill half the tube then count cells based on density prior to centrifugation.
4. Centrifuge the cell suspension and resuspend the pellet in fresh medium.
5. Add 10K cells per well with fresh growth medium (180 ul per well)
6. Centrifuge, if necessary, at 1000 rpm for 2-3 minutes with low deceleration. Centrifugation helps the cells to come together, if needed.
7. When desirable tight clusters are formed (3-5 days), proceed with the Seahorse XF assay.
8. For HepG2, DMEM PH 7.4 media from Seahorse supplemented with glutamine, sodium pyruvate and glucose were used (same concentration as the growth medium).
9. With a multiple channel, pull out about 150 microliters of spent medium and add 180 microliters of fresh Seahorse medium slowly sliding through the walls.
10. Follow with 2-3 washes to dilute out the FBS from the wells.
11. After every wash check if the spheroid is still in the center. The white spot of the spheroid is easily seen on dark lab counter tops.
12. Follow known protocol for traditional oxygen consumption rate (OCR) assay based on the mitochondrial stress test described in detail above.
13. Take before and after images to verify the spheroids remain centered throughout.
14. The DMEM contains high glucose for Panc1 cells as opposed to low glucose for HepG2.

### Results and Observations

As seen in FIG. 11, the spheroids generated in Agilent's plate looked very similar to the those generated in Corning and Insphero plates but with improved centering and more uniformity in terms of geometry. The size differences in the spheroids reflects the different geometries of the various ULA plates.

Referring now to FIG. 13, the oxygen consumption rate assay was performed using the mitochondrial stress test described above using Panc1 spheroids generated in a Corning plate and a plate with the geometric configuration of the present disclosure. The difference in oxygen consumption rate is attributed to the spheroids in the plates of the present disclosure having a diameter of about 150 micrometers to about 200 micrometers compared to the spheroids grown in the Corning plate having a diameter of about 200 micrometers to about 250 micrometers with a more pancake-like geometry as shown in FIG. 11.

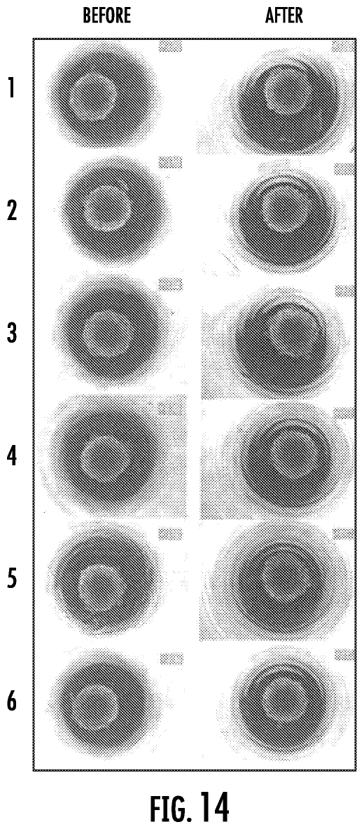

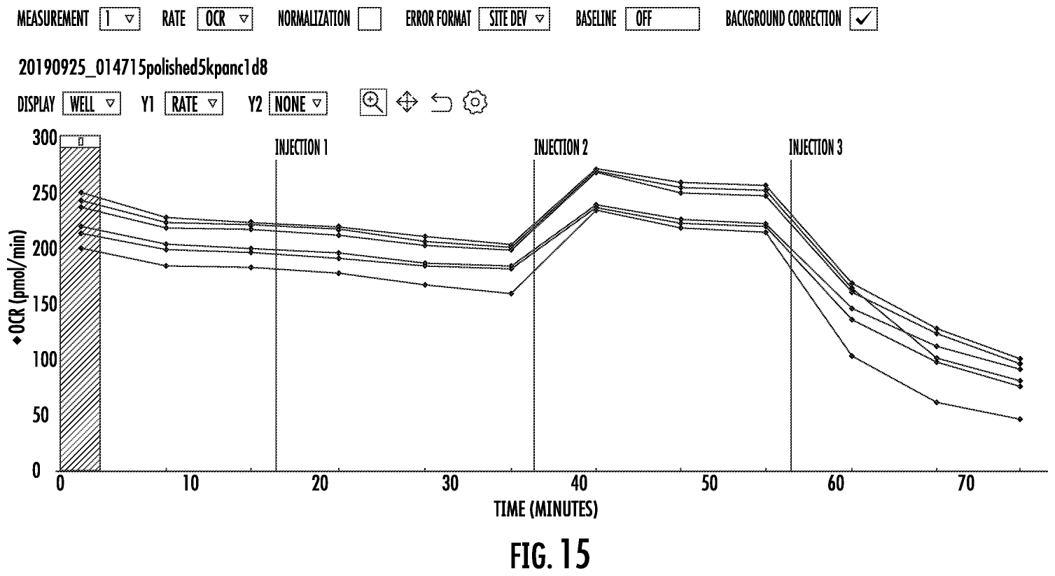

Next, and referring to FIGS. 14 and 15, the oxygen consumption rate was performed on wells 1-6 using the mitochondrial stress test described above using Panc1 spheroids generated and assayed after 8 days in a plate with the geometric configuration of the present disclosure. The spheroids showed good response to FCCP and antimycin A/rotenone that is typically seen in 2D Panc1 cells as well. Note that the Panc1 cells in the lab were resistant to oligomycin and hence did not show the dip on oxygen consumption. The plate had a polished surface with 20 microliters of Lipidure coating.

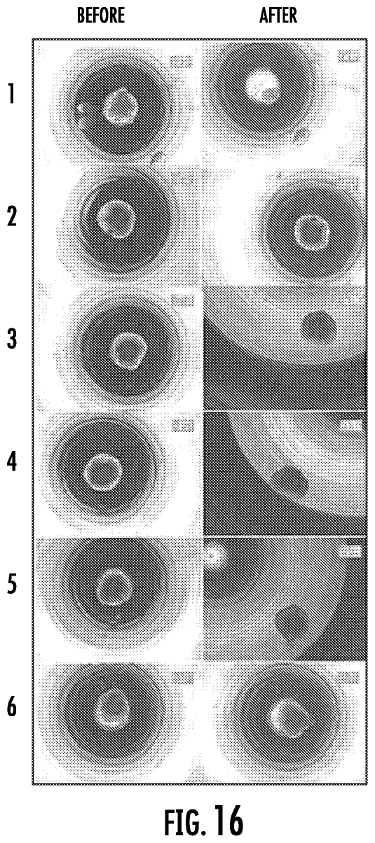

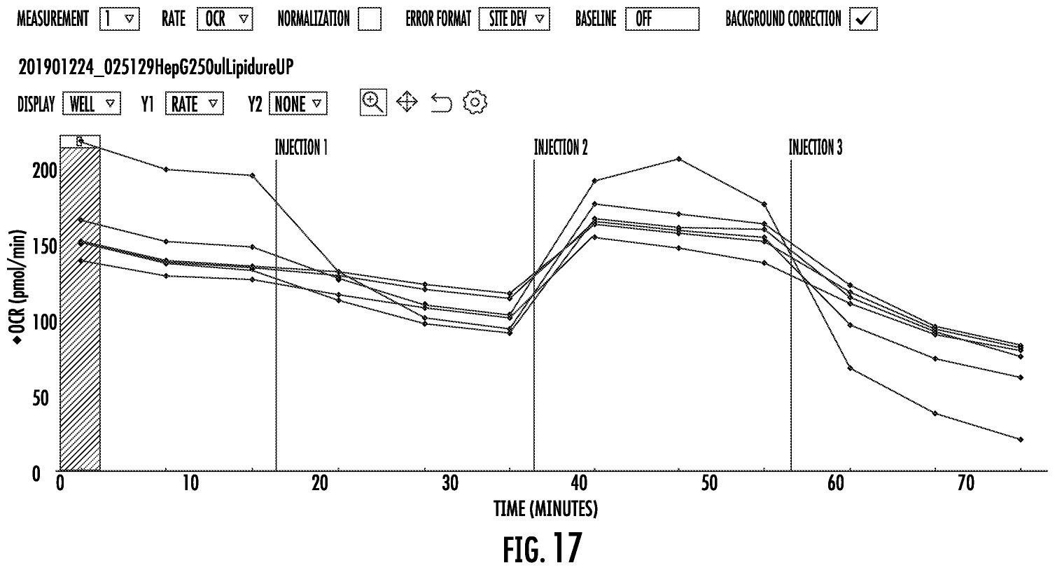

Further, and referring to FIGS. 16 and 17, the oxygen consumption rate was performed on wells 1-6 using the mitochondrial stress test described above using HepG2 spheroids generated and assayed after 6 days in a plate with the geometric configuration of the present disclosure. The spheroids showed good response to oligomycin, FCCP and antimycin A/rotenone that is typically seen in 2D HepG2 cells as well. The plate had an unpolished surface with 50 microliters of Lipidure coating. In FIG. 16, the spheroids in well 1, 3, 4 and 5 seemed to have moved away from the center after the assay. However as seen in FIG. 17, it can be easily deciphered that the displacement happened post assay while the sensor cartridge was removed from the plate.

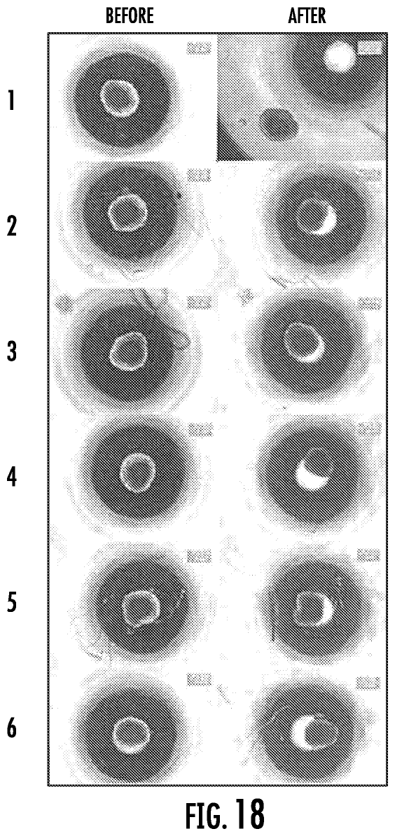

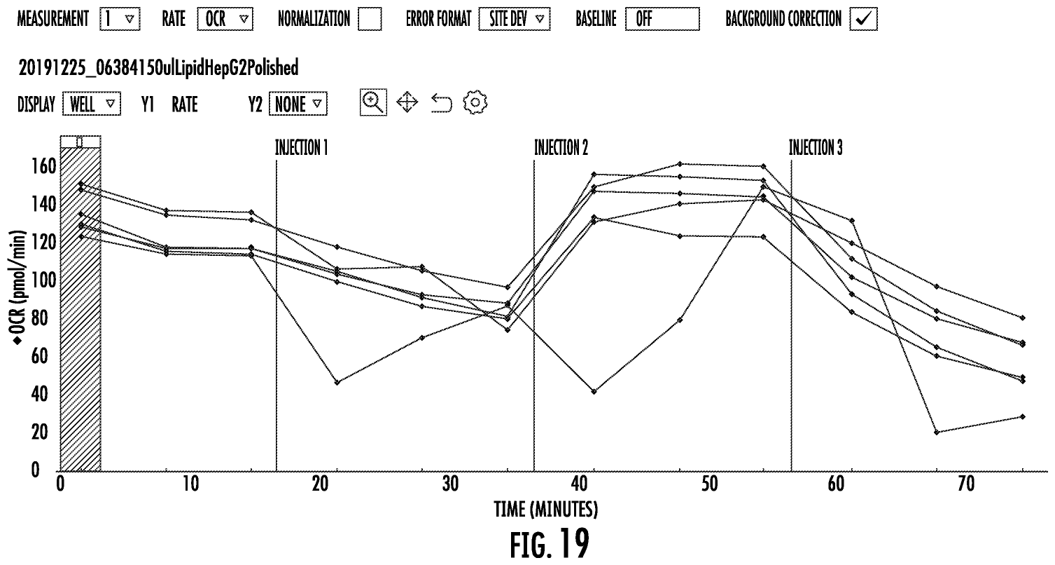

Lastly, and referring to FIGS. 18 and 19, the oxygen consumption rate was performed on wells 1-6 using the mitochondrial stress test described above using HepG2 spheroids generated and assayed after 6 days in a plate with the geometric configuration of the present disclosure. The spheroids showed good response to oligomycin, FCCP and antimycin A/rotenone that is typically seen in 2D HepG2 cells as well. The plate had a polished surface with 50 microliters of Lipidure coating.

### Example 2

Panc1, HepG2, and C2C12 cell lines were used to form spheroids in an apparatus with wells having the geometry shown at least in FIGS. 2, 3, and 4 of the present disclosure. The wells were coated with either Lipidure® or BioFLOAT® ultra low adhesion coatings according to manufacturer protocols, after which the cell lines were introduced to the well plates and spheroids were formed according to the following protocol:
Apply ULA coatings according to manufacturer's protocols
Use trypsin to detach cells of interest (C2C12, Panc1, or HepG2) and create a single cell suspension
Deactivate trypsin via addition of complete culture media
Perform a cell count
Centrifuge the cell suspension at 250 G for 5 minutes and resuspend the pellet in fresh medium at the required density.
Add 300-1500 cells per well with fresh growth medium to a final volume of 200 ul per well
Centrifuge, if necessary, at 250 G for 5 minutes with low deceleration. Centrifugation helps the cells to come together, if needed.
When desirable tight clusters are formed (typically 3-5 days), proceed with the Seahorse XF assay.
DMEM PH 7.4 media from Seahorse supplemented with glutamine, sodium pyruvate and glucose were used (same concentration as the growth medium).
With a multiple channel, pull out about 100 microliters of spent medium and add 100 microliters of fresh Seahorse medium slowly sliding through the walls.
Follow with 3-5 washes to dilute out the old media from the wells.
Perform desired Seahorse assay-Basal, Mito Stress Test, FCCP addition, or others.

The resulting spheroids remained centered in the wells during performance of assays using the SEAHORSE analysis platform, and a basal metabolic signal could be detected. In addition, spheroids of various sizes could be generated, and the generation of one spheroid per well was possible without using centrifugation, as evidenced by the figures and graphs discussed below.

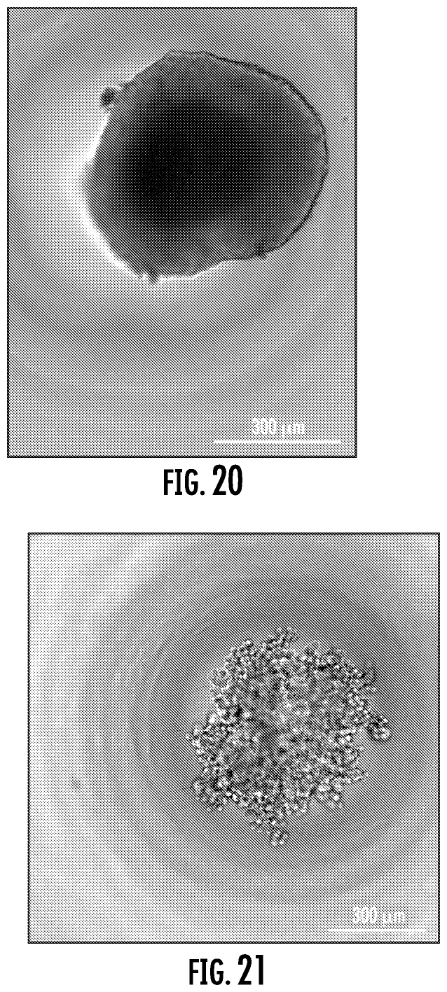

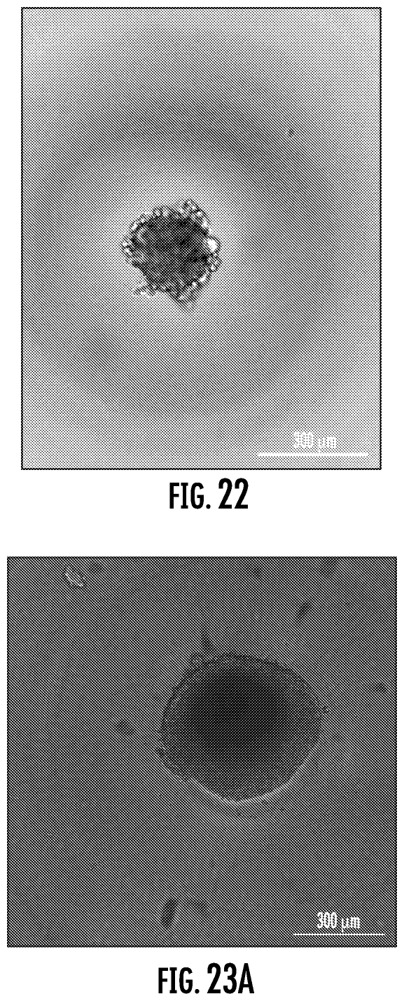

Specifically, FIGS. 20, 21 and 22 show the successful generation of spheroids in the wells of the plates contemplated by the present disclosure using three different cell lines. For instance, FIG. 20 is a top view photograph of a HepG2 spheroid cultured in the apparatus contemplated by the present disclosure, where the spheroid was generated using centrifugation. Meanwhile, FIG. 21 is a top view photograph of a Panc1 spheroid cultured in the apparatus contemplated by the present disclosure, where the spheroid was generated using centrifugation. Further, FIG. 22 is a top view photograph of a C2C12 spheroid cultured in the apparatus contemplated by the present disclosure, where the spheroid was generated using centrifugation.

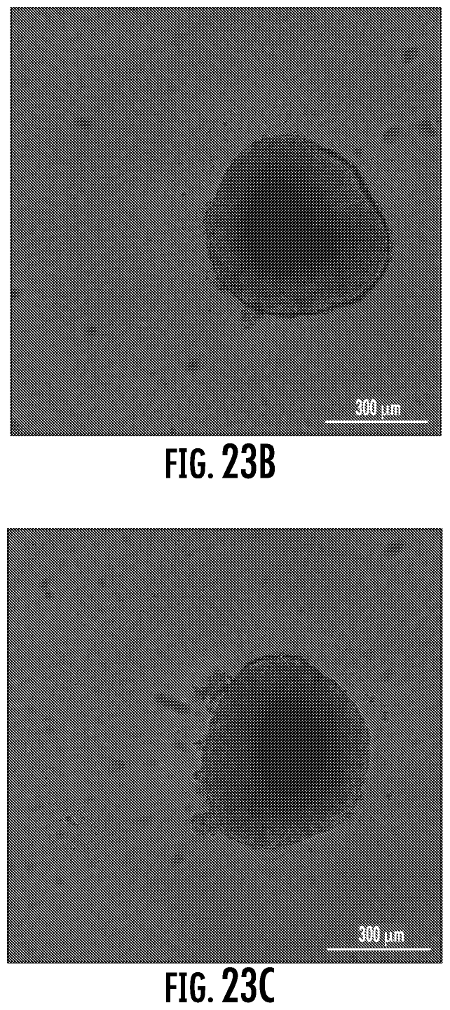

Next, FIGS. 23A, 23B, and 23C show HepG2 spheroids cultured in the apparatus contemplated by the present disclosure, where the apparatus was coated with a 2-methacryloyloxyethyl phosphorylcholine polymer or MPC polymer (e.g., Lipidure®), followed by centrifugation.

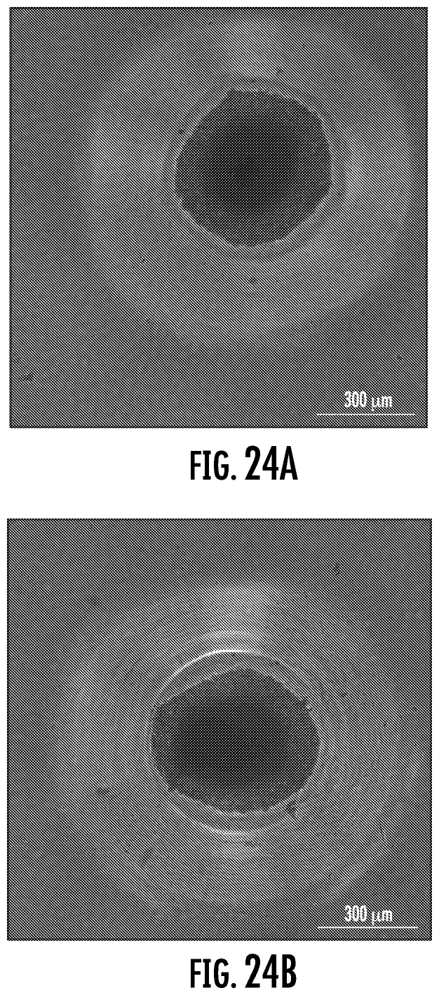

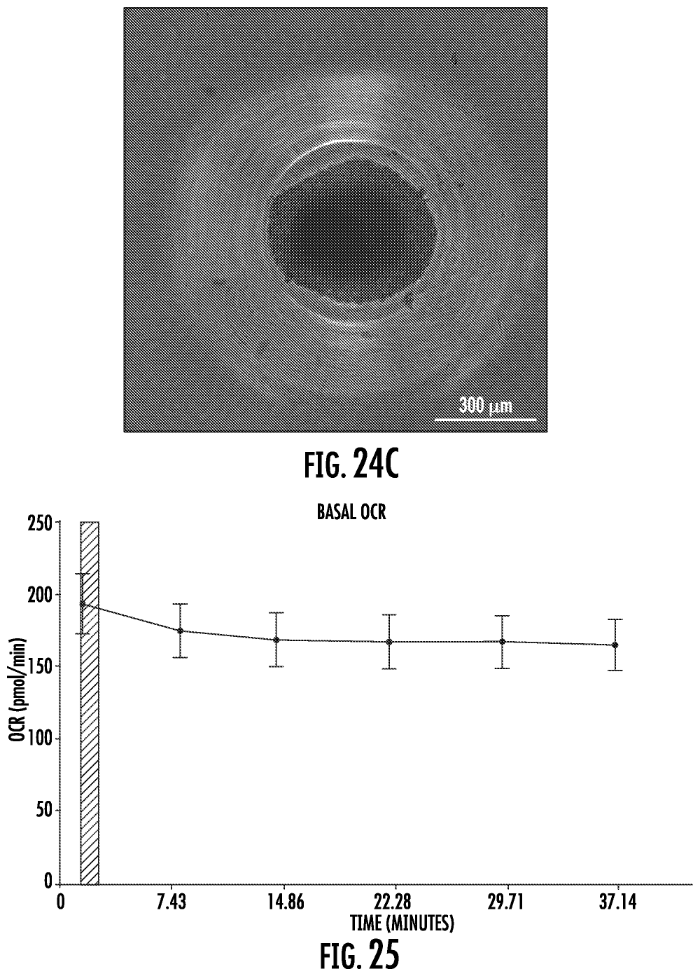

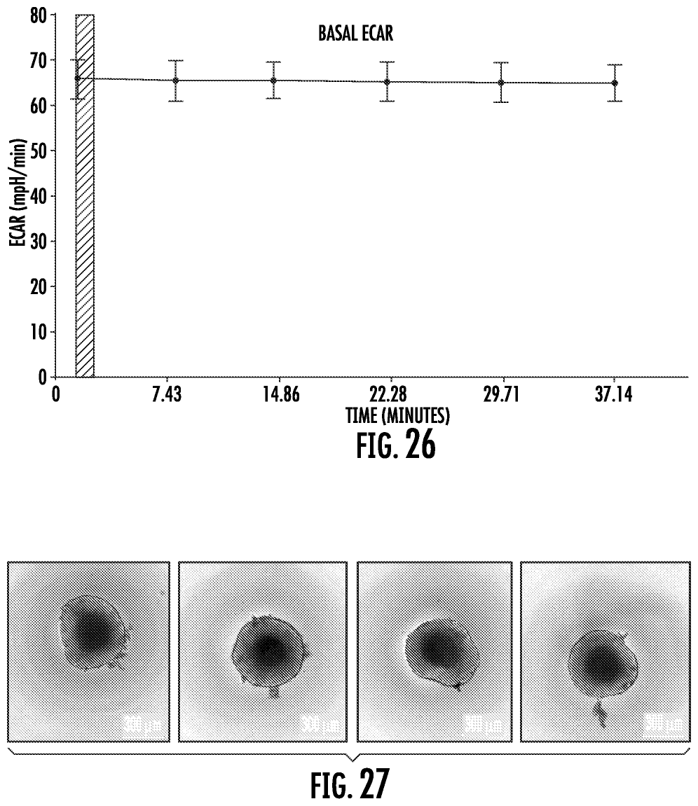

Further, FIGS. 24A, 24B, and 24C show HepG2 spheroids cultured in the apparatus contemplated by the present disclosure, where the apparatus was coated with BioFLOAT®, followed by centrifugation. FIG. 25 is a graph showing the oxygen consumption rate (OCR) of spheroids formed from HepG2 cells cultured in the apparatus contemplated by the present disclosure with a BioFLOAT® coating, followed by centrifugation, while FIG. 26 is a graph showing the extra cellular acidification rate (ECAR) of a spheroid formed from HepG2 cells cultured in the apparatus contemplated by the present disclosure with a BioFLOAT® coating, followed by centrifugation. These graphs show that basal metabolic signals are measurable when spheroids are cultured in wells of the apparatus contemplated by the present disclosure. Further, FIG. 27 is a series of top view photographs of various HepG2 spheroids after the OCR and ECAR assays summarized in FIGS. 25 and 26 were completed, and the photographs demonstrate that the spheroids remained centered during the metabolic assay.

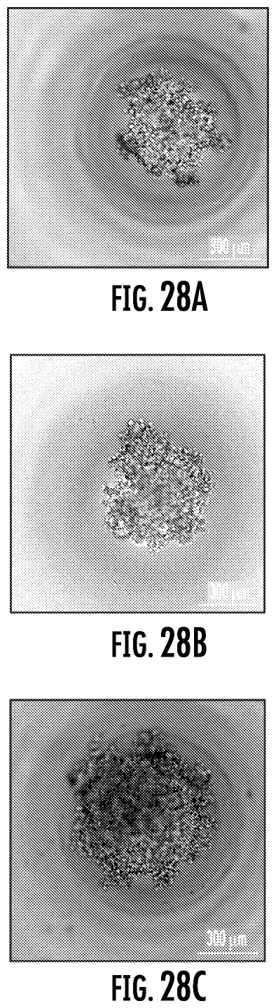

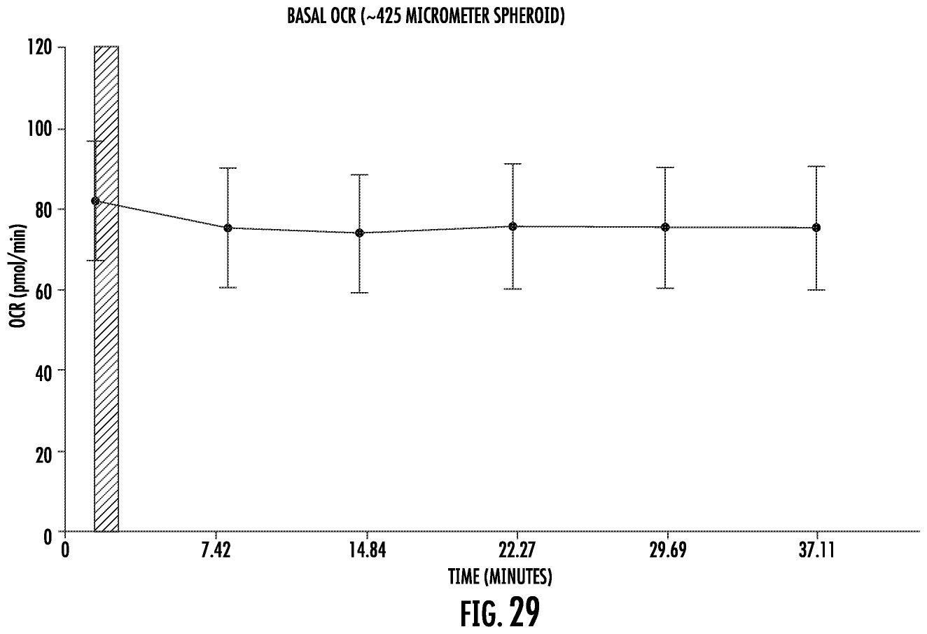

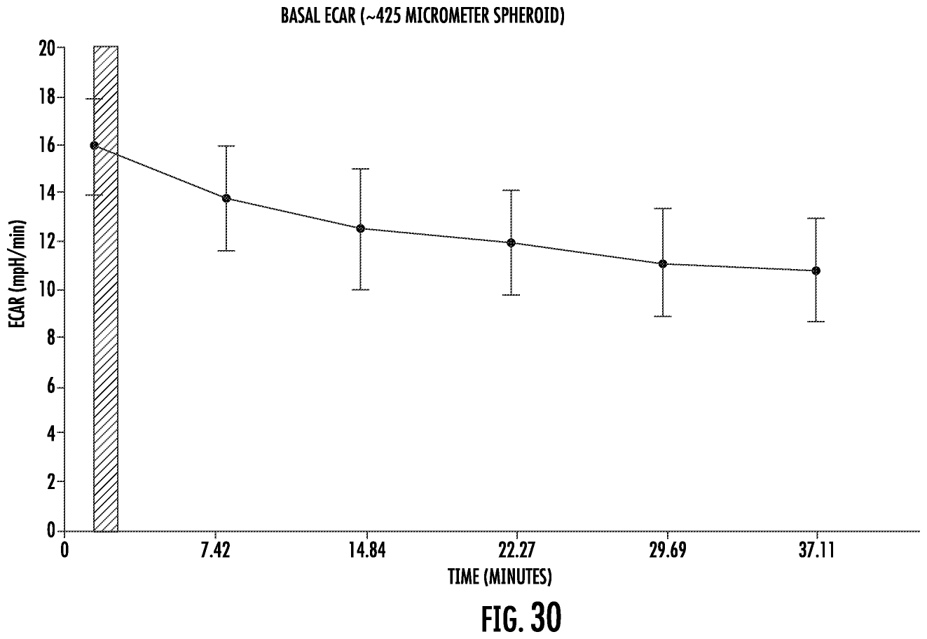

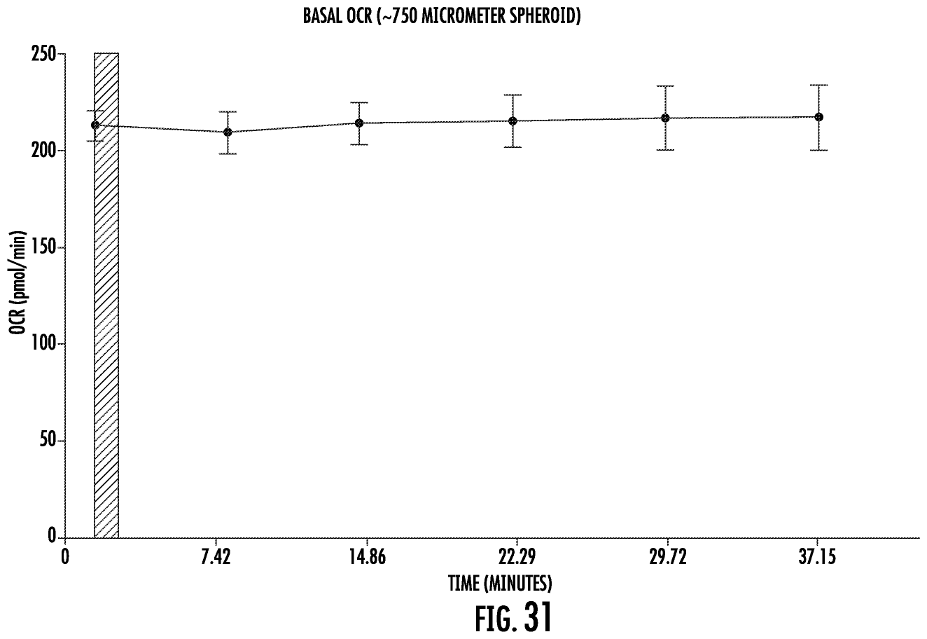

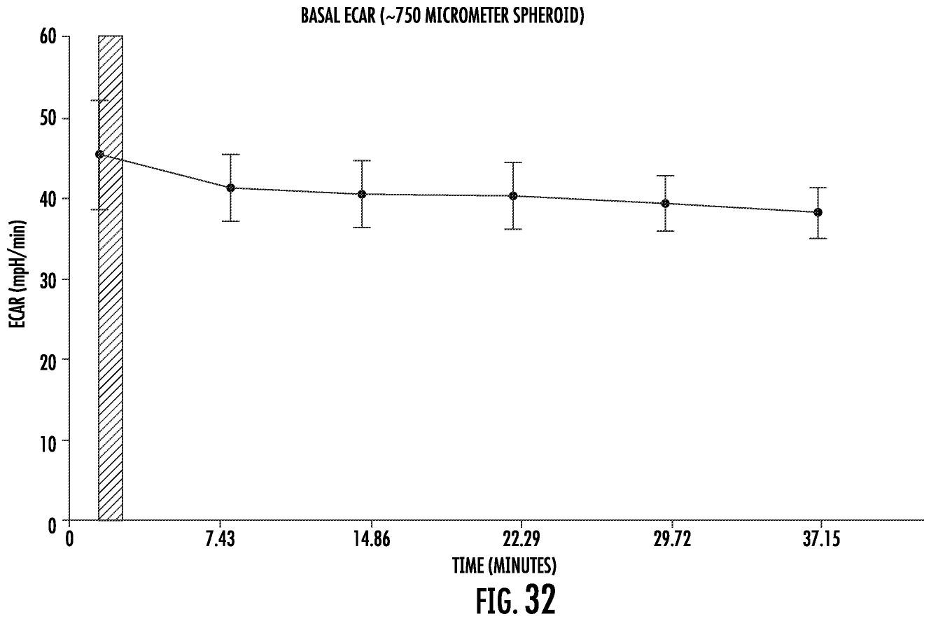

Additionally, FIGS. 28A, 28B, and 28C demonstrate that spheroids of different sizes can be generated, while FIGS. 29, 30, 31, and 32 demonstrate that basal metabolic signals can be measured for Panc1 spheroids cultured in the apparatus contemplated by the present disclosure. For example, FIG. 28A shows a Panc1 spheroid cultured in the apparatus contemplated by the present disclosure, where the apparatus was coated with BioFLOAT®, followed by centrifugation, where the spheroid has a diameter of about 425 micrometers. In addition, FIG. 28B shows a Panc1 spheroid cultured in the apparatus contemplated by the present disclosure, where the apparatus was coated with BioFLOAT®, followed by centrifugation, where the spheroid has a diameter of about 550 micrometers. Next, FIG. 28C shows a Panc1 spheroid cultured in the apparatus contemplated by the present disclosure, where the apparatus was coated with BioFLOAT®, followed by centrifugation, where the spheroid has a diameter of about 750 micrometers. Further, FIG. 29 is a graph showing the oxygen consumption rate (OCR) of a spheroid formed from Panc1 cells cultured in the apparatus contemplated by the present disclosure with a BioFLOAT® coating, followed by centrifugation, where the spheroid has a diameter of about 425 micrometers, while FIG. 30 is a graph showing the extra cellular acidification rate (ECAR) of spheroids formed from Panc1 cells cultured in the apparatus contemplated by the present disclosure with a BioFLOAT® coating, followed by centrifugation, where the spheroid has a diameter of about 425 micrometers. In addition, FIG. 31 is a graph showing the oxygen consumption rate (OCR) of a spheroid formed from Panc1 cells cultured in the apparatus contemplated by the present disclosure with a BioFLOAT® coating, followed by centrifugation, where the spheroid has a diameter of about 750 micrometers, while FIG. 32 is a graph showing the extra cellular acidification rate (ECAR) of spheroids formed from Panc1 cells cultured in the apparatus contemplated by the present disclosure with a BioFLOAT® coating, followed by centrifugation, where the spheroid has a diameter of about 750 micrometers.

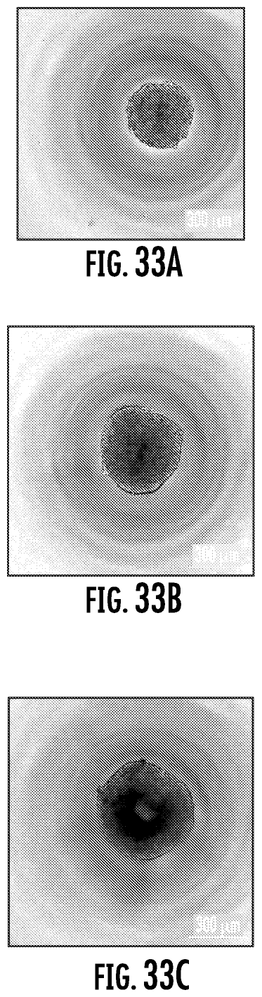

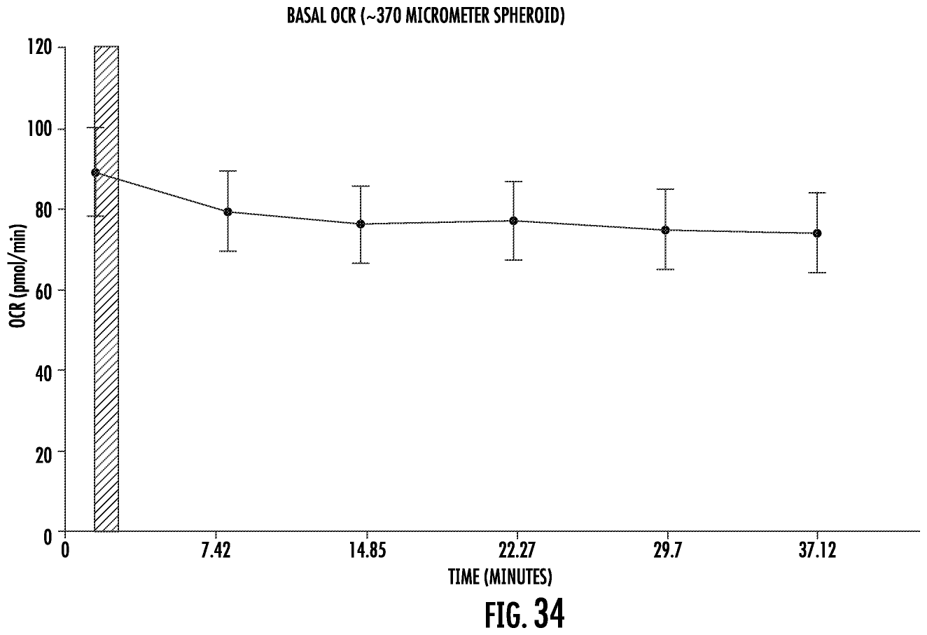

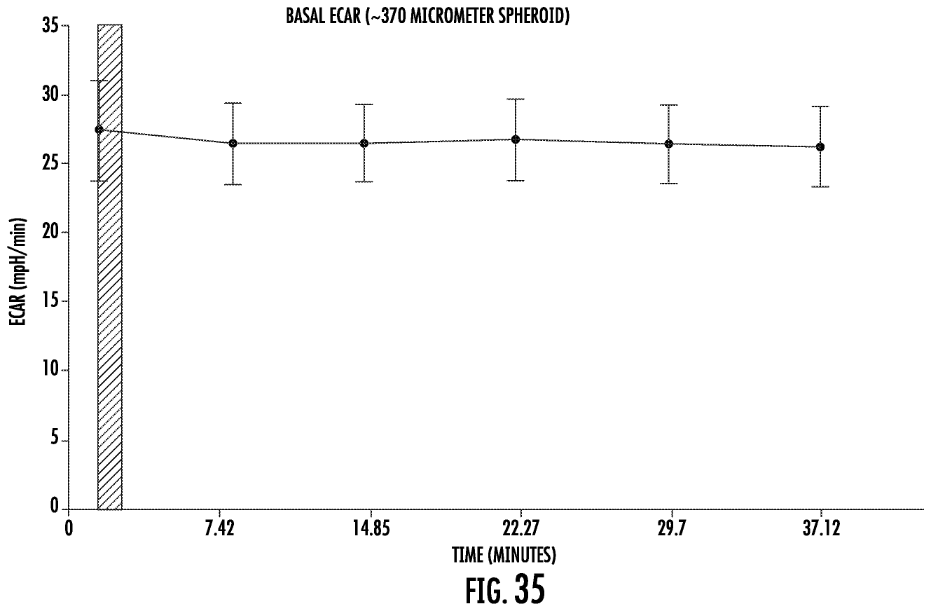

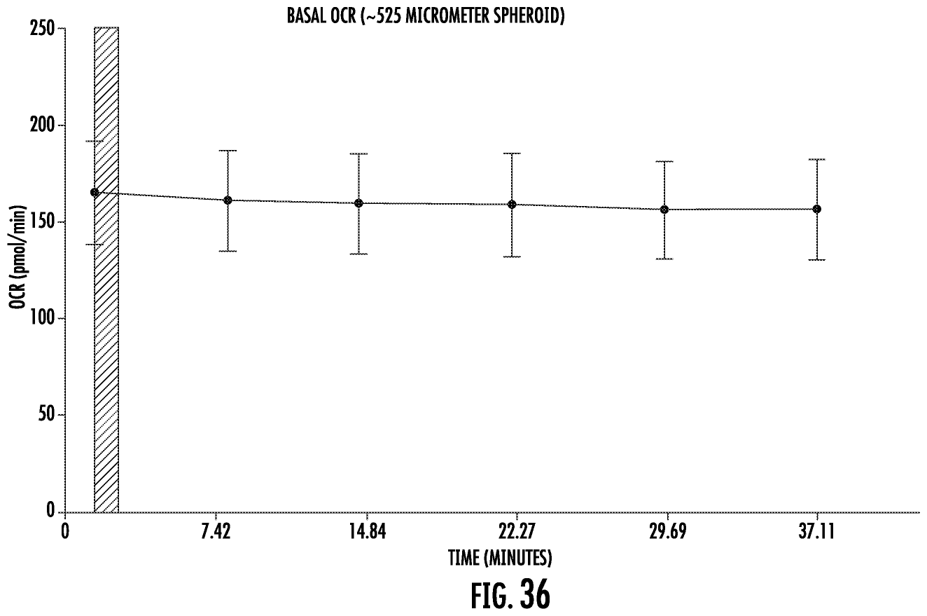

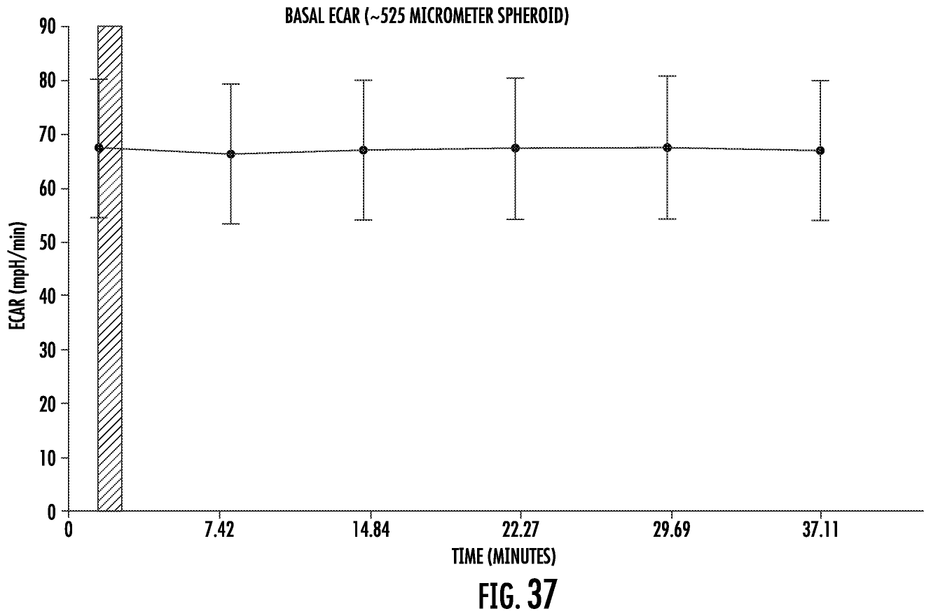

Additionally, FIGS. 33A, 335B, and 33C demonstrate that spheroids of different sizes can be generated, while FIGS. 34, 35, 36, and 37 demonstrate that basal metabolic signals can be measured for HepG2 spheroids cultured in the apparatus contemplated by the present disclosure. For example, FIG. 33A shows a HepG2 spheroid cultured in the apparatus contemplated by the present disclosure, where the apparatus was coated with BioFLOAT®, followed by centrifugation, where the spheroid has a diameter of about 370 micrometers. In addition, FIG. 33B shows a HepG2 spheroid cultured in the apparatus contemplated by the present disclosure, where the apparatus was coated with BioFLOAT®, followed by centrifugation, where the spheroid has a diameter of about 450 micrometers. Next, FIG. 33C shows a HepG2 spheroid cultured in the apparatus contemplated by the present disclosure, where the apparatus was coated with BioFLOAT®, followed by centrifugation, where the spheroid has a diameter of about 525 micrometers. Further, FIG. 34 is a graph showing the oxygen consumption rate (OCR) of a spheroid formed from HepG2 cells cultured in the apparatus contemplated by the present disclosure with a BioFLOAT® coating, followed by centrifugation, where the spheroid has a diameter of about 370 micrometers, while FIG. 35 is a graph showing the extra cellular acidification rate (ECAR) of a spheroid formed from HepG2 cells cultured in the apparatus contemplated by the present disclosure with a BioFLOAT® coating, followed by centrifugation, where the spheroid has a diameter of about 370 micrometers. In addition, FIG. 36 is a graph showing the oxygen consumption rate (OCR) of a spheroid formed from HepG2 cells cultured in the apparatus contemplated by the present disclosure with a BioFLOAT® coating, followed by centrifugation, where the spheroid has a diameter of about 525 micrometers, while FIG. 37 is a graph showing the extra cellular acidification rate (ECAR) of a spheroid formed from HepG2 cells cultured in the apparatus contemplated by the present disclosure with a BioFLOAT® coating, followed by centrifugation, where the spheroid has a diameter of about 525 micrometers.

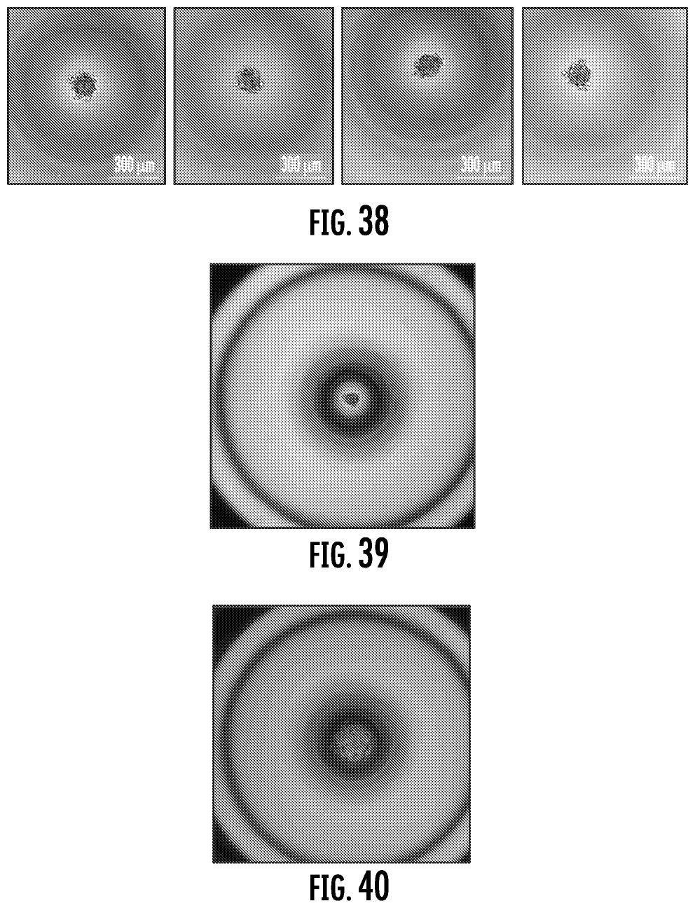

The present example also demonstrates that smaller spheroids can be formed in the wells of the apparatus contemplated by the present disclosure. For instance, FIG. 38 shows a series of top view photographs of various C2C12 spheroids cultured in the apparatus contemplated by the present disclosure, where the apparatus was coated with BioFLOAT®, where the spheroids have a diameter of about 150 micrometers.

Moreover, the present example also demonstrates that the wells of the apparatus of the present disclosure facilitate the formation of a single spheroid per well in the absence of centrifugation. For example, FIG. 39 is a top view photograph of a C2C12 spheroid cultured in the apparatus contemplated by the present disclosure, where the apparatus was coated with BioFLOAT®, without centrifugation; and FIG. 40 is a top view photograph of a Panc1 spheroid cultured in the apparatus contemplated by the present disclosure, where the apparatus was coated with BioFLOAT®, without centrifugation.

### Example 3

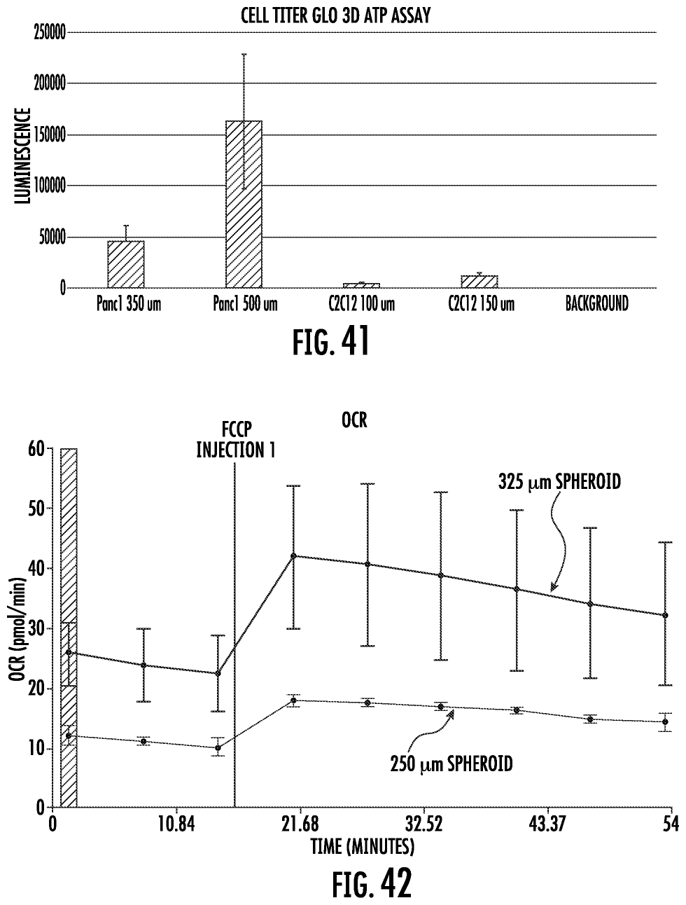

First, C2C12 and Panc1 cells were plated in spheroid plates coated with BioFLOAT® as contemplated by the present disclosure at either 600 cells/well (for smaller spheroids) or 1200 cells/well (for larger spheroids). No centrifugation was used, and cells were allowed to form spheroids over 3 days. On day 3, ATP levels were measured with the Promega Cell Titer Glo 3D kit for various sized spheroids. FIG. 41 is a bar graph illustrating that ATP levels are proportional to spheroid size. In particular, the larger the spheroid, the higher the luminescence value and thus ATP level, for both C2C12 and Panc1 cell types.

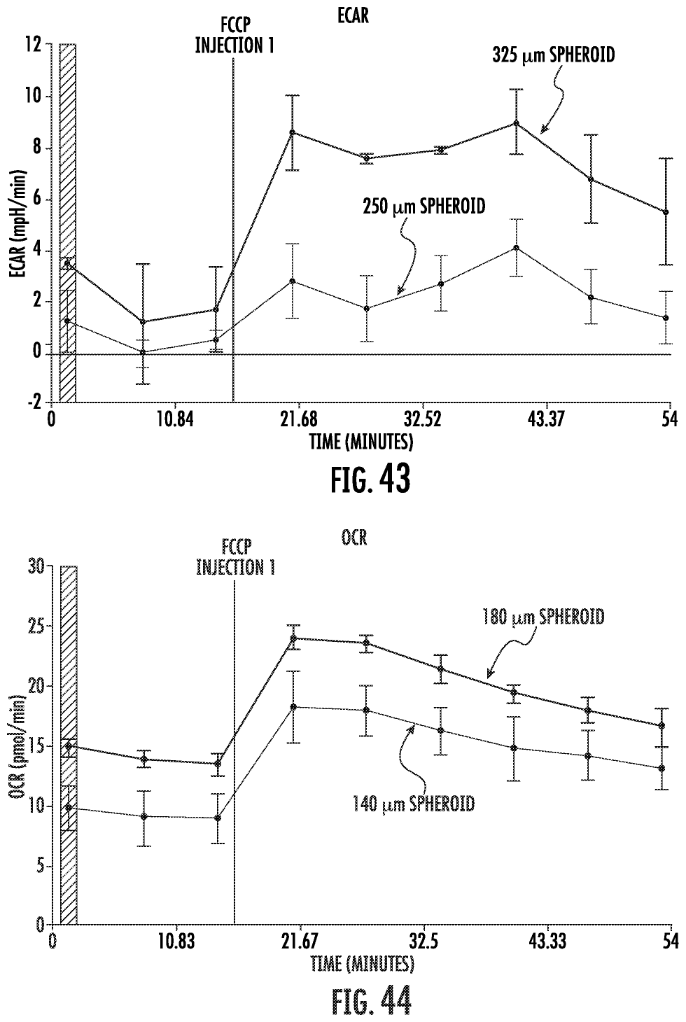

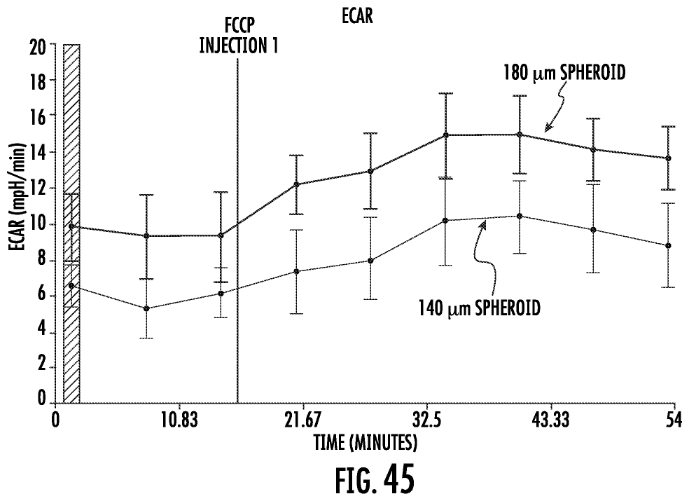

Next, C2C12 and Panc1 cells were seeded in BioFLOAT® coated spheroid plates as contemplated by the present disclosure at two different concentrations. The cells were allowed to settle without centrifugation and form spheroids for two days. OCR and ECAR assays were then run including both basal measurements and FCCP injection (final concentration 1 μM), where FCCP, which is an uncoupling agent that collapses the proton gradient and disrupts the mitochondrial membrane potential resulting in the electron transport chain being uninhibited, was used to cause maximal respiration in the cells. The largest cross-sectional diameter was measured to characterize the size of the spheroids. Panc1 spheroids were roughly 325 micrometers in diameter for the larger spheroids and roughly 250 micrometers in diameter for the smaller spheroids, while the large C2C12 spheroids were roughly 180 micrometers in diameter and the small C2C12 spheroids were roughly 140 micrometers in diameter. FIG. 42 is a graph showing the oxygen consumption rate (OCR) of spheroids formed from Panc1 cells, where the spheroids had diameters of about 325 micrometers and about 250 micrometers. FIG. 43 is a graph showing the extra cellular acidification rate (ECAR) of spheroids formed from Panc1, where the spheroids had diameters of about 325 micrometers and about 250 micrometers. FIG. 44 is a graph showing the oxygen consumption rate (OCR) of spheroids formed from C2C12 cells, where the spheroids had diameters of about 180 micrometers and about 140 micrometers. FIG. 45 is a graph showing the extra cellular acidification rate (ECAR) of spheroids formed from C2C12 cells, where the spheroids had diameters of about 180 micrometers and about 140 micrometers. As shown, the larger spheroids for both cell types had higher OCR and ECAR compared to the smaller spheroids for both cell types, and both the OCR and ECAR increased after FCCP injection.

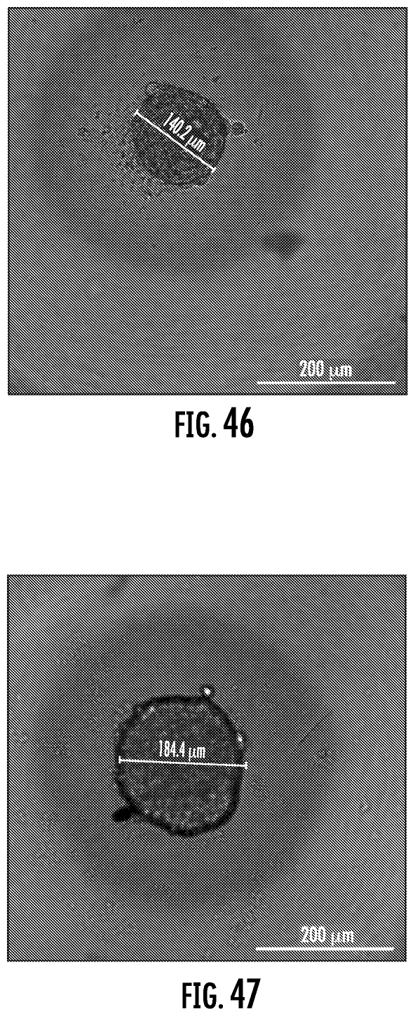

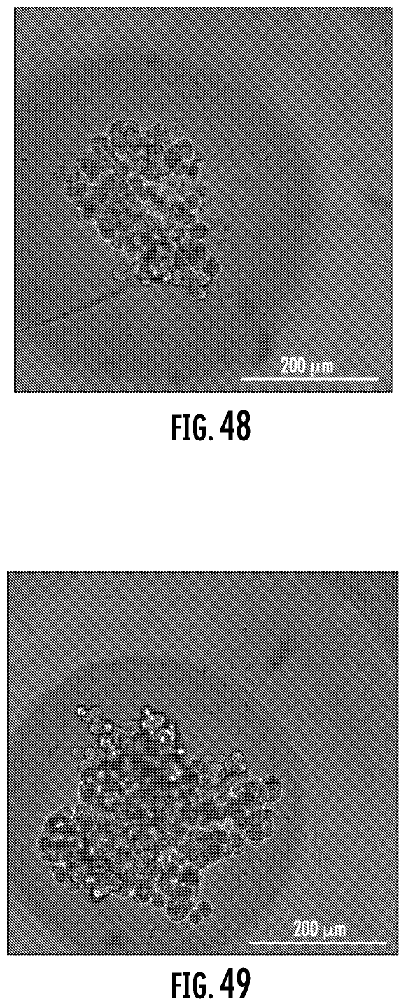

After completing on the OCR and ECAR assays, spheroids were imaged using brightfield imaging on a BioTek Cytation 1 imager, as shown in FIGS. 46-49. FIG. 46 is an image of the 140 micrometer spheroids assayed to determine the OCR and ECAR of FIGS. 44 and 45. FIG. 47 is an image of the 180 micrometer spheroids assayed to determine the OCR and ECAR of FIGS. 44 and 45. FIG. 48 is an image of the 250 micrometer spheroids assayed to determine the OCR and ECAR of FIGS. 42 and 43. FIG. 49 is an image of the 325 micrometer spheroids assayed to determine the OCR and ECAR of FIGS. 42 and 43.

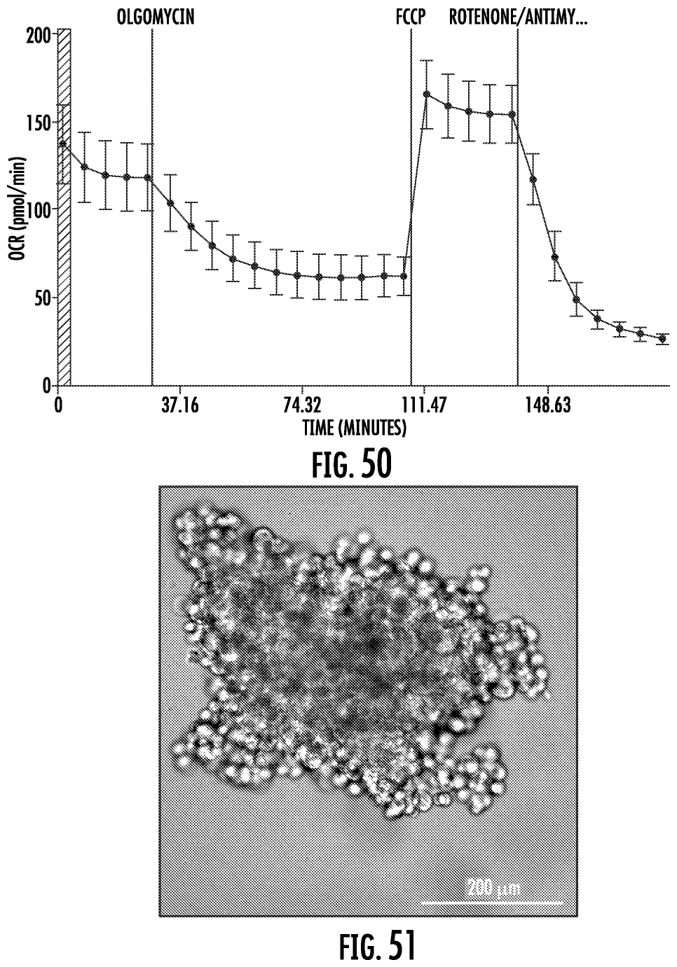

Next, successful growth and measurement of spheroids in the same plate was demonstrated. Panc1 spheroids were grown in a BioFLOAT® coated spheroid plate as contemplated by the present disclosure until reaching a size of roughly 400 micrometers in diameter. Media was changed to Seahorse DMEM on the day of the assay. Plates were moved to a non-CO₂, 37° C. incubator for 1 hour. A Mito Stress Test was run including Oligomycin, FCCP, and Rotenone/Antimycin injections. FIG. 50 is a graph showing the oxygen consumption rate (OCR) of the spheroids formed from Panc1 cells cultured in the apparatus contemplated by the present disclosure at each injection point and thereafter. As shown, additional measurement cycles were added to give time for the drugs to diffuse into the spheroids. The spheroids responded well to all drugs and remained in position throughout the measurements. FIG. 51 is an image of the 400 micrometer spheroids assayed to determine the OCR shown in FIG. 50.

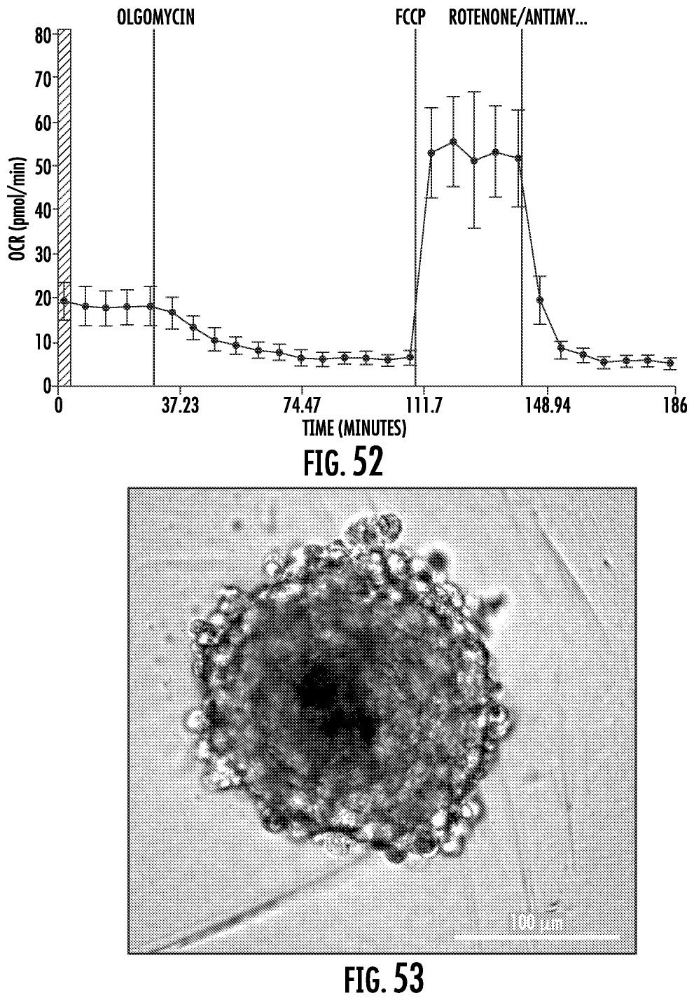

Next, successful growth and measurement of spheroids in the same plate was demonstrated. C2C12 spheroids were grown in a BioFLOAT® coated spheroid plate as contemplated by the present disclosure until reaching a size of roughly 180 micrometers in diameter. Media was changed to Seahorse DMEM on the day of the assay. Plates were moved to a non-CO₂, 37° C. incubator for 1 hour. A Mito Stress Test was run including Oligomycin, FCCP, and Rotenone/Antimycin injections. FIG. 52 is a graph showing the oxygen consumption rate (OCR) of the spheroids formed from C2C12 cells cultured in the apparatus contemplated by the present disclosure at each injection point and thereafter. As shown, additional measurement cycles were added to give time for the drugs to diffuse into the spheroids. The spheroids responded well to all drugs and remained in position throughout the measurements. FIG. 53 is an image of the 180 micrometer spheroids assayed to determine the OCR shown in FIG. 52.

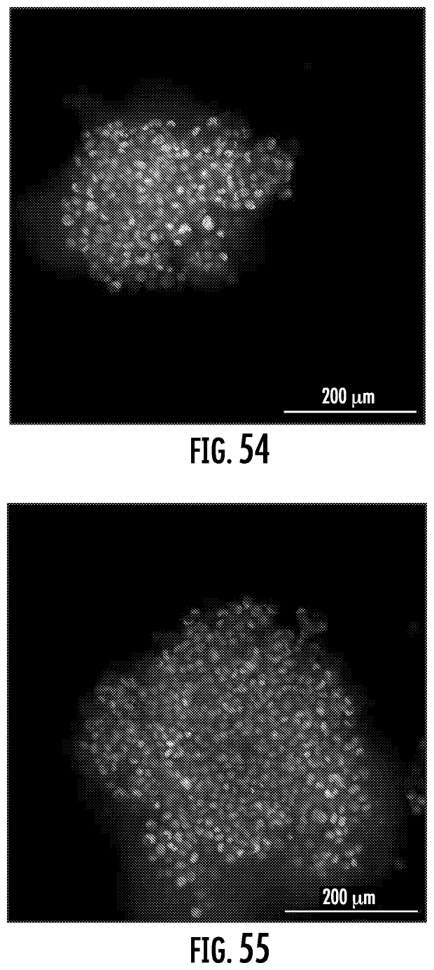

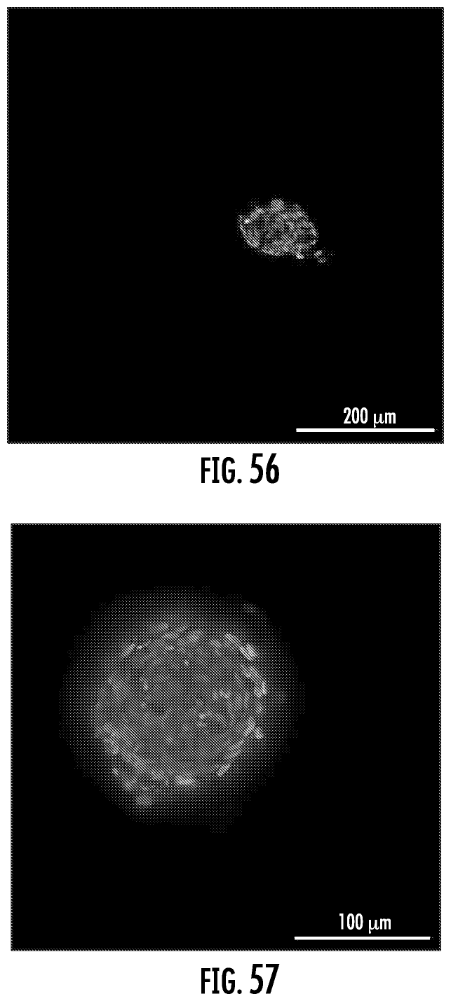

In addition, spheroids of two different cell types at different cell numbers were grown in BioFLOAT® coated spheroid plates as contemplated by the present disclosure. Live spheroids were then stained with CyQuant Direct Cell Proliferation Dye and imaged by confocal on the BioTek Cytation 10. As shown in FIGS. 54-57, successful staining shows cells are viable and also confirms the suitability of spheroids and the plates contemplated by the present disclosure for confocal imaging purposes. Specifically, FIG. 54 is a confocal image of a spheroid formed from an initial seeding of 600 Panc1 cells cultured in the apparatus contemplated by the present disclosure with a BioFLOAT® coating, where the spheroid had a diameter of about 350 micrometers; FIG. 55 is a confocal image of a spheroid formed from an initial seeding of 1200 Panc1 cells cultured in the apparatus contemplated by the present disclosure with a BioFLOAT® coating, where the spheroid had a diameter of about 500 micrometers; FIG. 56 is a confocal image of a spheroid formed from an initial seeding of 600 C2C12 cells cultured in the apparatus contemplated by the present disclosure with a BioFLOAT® coating, where the spheroid had a diameter of about 100 micrometers; and FIG. 57 is a confocal image of a spheroid formed from an initial seeding of 1200 C2C12 cells cultured in the apparatus contemplated by the present disclosure with a BioFLOAT® coating, where the spheroid had a diameter of about 150 micrometers.

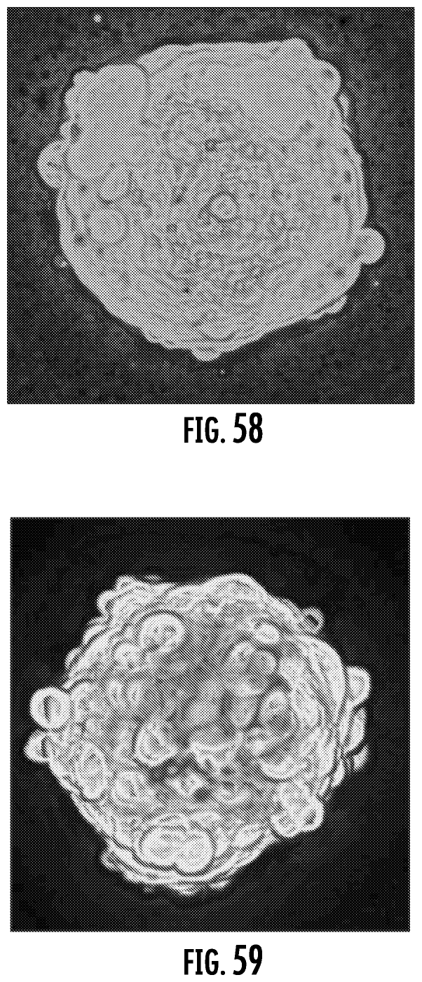

Further, additional fluorophores were used to characterize live C2C12 spheroids grown in spheroid plates coated with a BioFLOAT® coating as contemplated by the present disclosure. Confocal imaging of both Cell Tracker Orange and Calcein AM enabled clear visualization of the live spheroids as shown with Z-projection images. Specifically, FIG. 58 is a confocal image of a spheroid formed from C2C12 cells cultured in the apparatus contemplated by the present disclosure with a BioFLOAT® coating and stained with Cell Tracker Orange, while FIG. 59 is a confocal image of a spheroid formed from C2C12 cells cultured in the apparatus contemplated by the present disclosure with a BioFLOAT® coating and stained with Calcein AM.

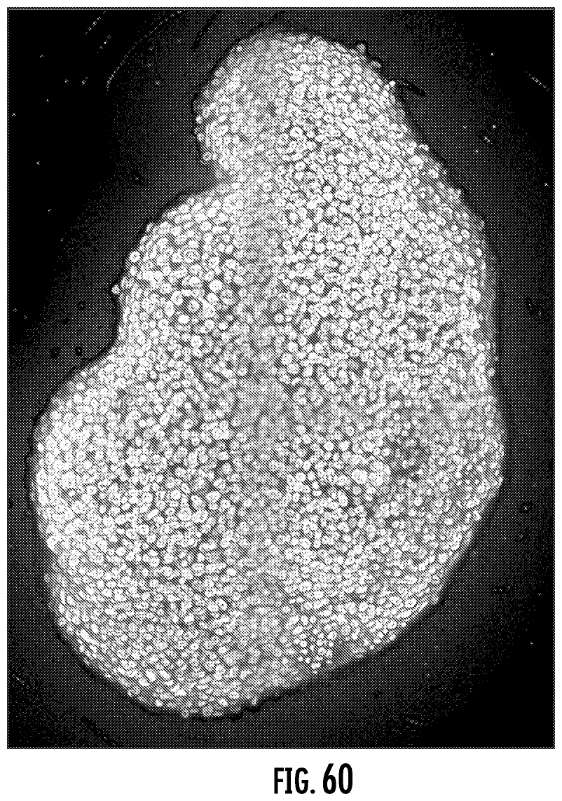

Lastly, FIG. 60, which is a confocal image of a spheroid formed from HepG2 cells cultured in the apparatus contemplated by the present disclosure with a BioFLOAT® coating and stained with Hoescht 34580, illustrates a tiled, stitched, z-projection image clearly depicting the ability to visualize individual nuclei in the live spheroid.

### Example 4

Spheroids can also be generated in other spheroid plates and transferred into the spheroid plate described here for measurement. This workflow has extra challenges to ensure that spheroids are not damaged or lost during the transfer process. Due to requiring visualization of the spheroid being transferred in the tip of the pipette, this protocol is challenging with very small spheroids that are difficult to visualize by eye. A centrifugation step after transfer is also required to generate usable data. Example transfer protocol:
Fill wells of plate described in this invention with 170 ul Seahorse media
Pipette 20 ul of media/spheroid from a plate containing pre-grown spheroids. Ensure that spheroid is in the tip of the pipette.
Allow spheroid to settle to tip by gravity.
Dispense the minimum amount of medium that enables the spheroid to be transferred to the new well.
Centrifuge at 200 G with low brake for 5 minutes, for higher quality data.
Incubate in a non-CO2 incubator at 37° C. for 1 hr
Run Seahorse assay

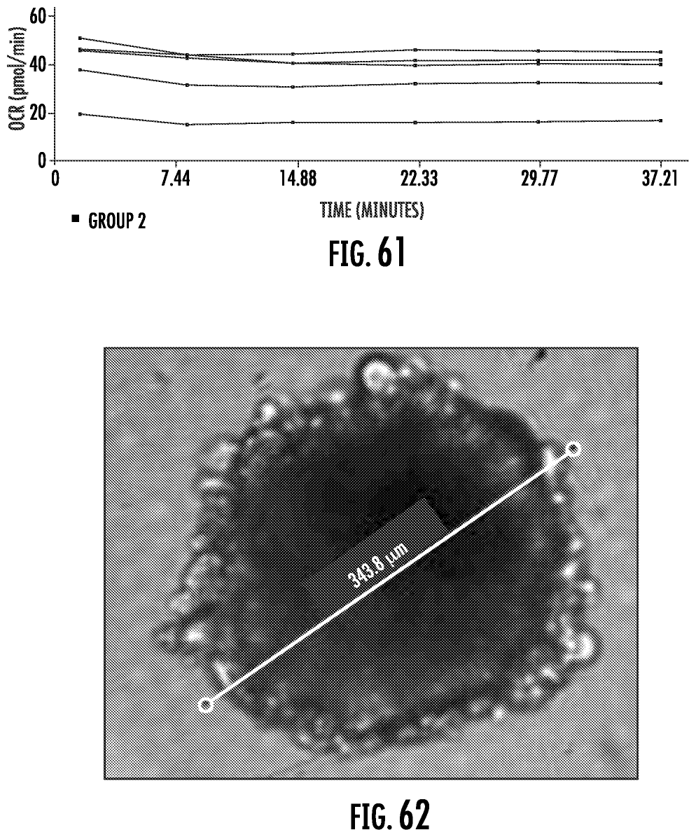

In order to assess transfer capabilities, liver spheroids (300 μm) were purchased from InSphero and maintained according to manufacturer protocols until time of assay. As shown in FIG. 61, basal OCR response is not consistent across all spheroids. In particular, one spheroid had a lower signal than the other spheroids (the line closest to the x-axis). As shown in FIG. 62, the spheroid was imaged after the Seahorse assay to assess morphology and was observed to have many loose cells around the outside of the spheroid suggesting it may have been damaged during the transfer process.

### Example 5

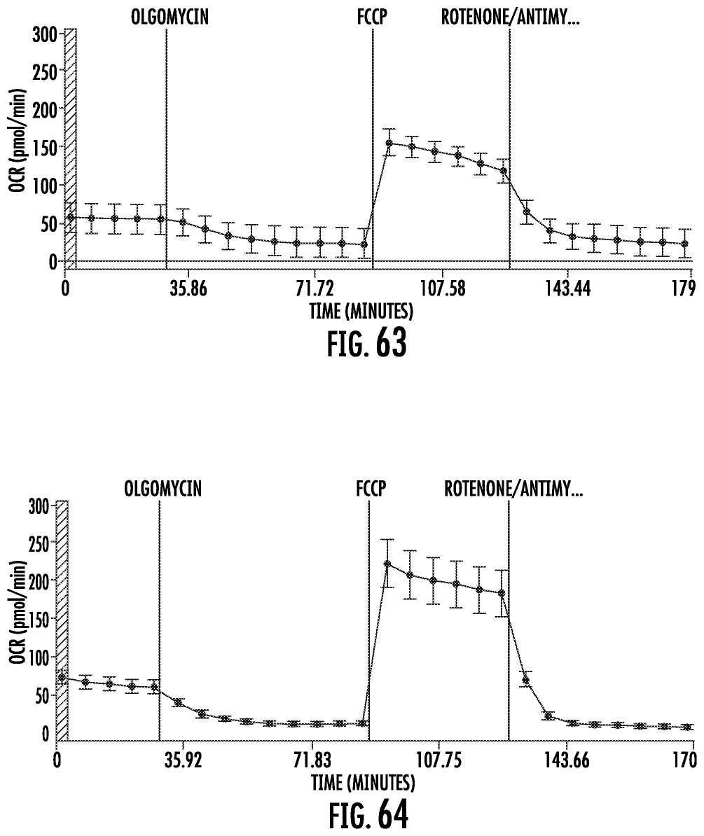

The plate described in this invention can be made out of a variety of materials, with the optimal material depending on the ultimate measurement application. Plates were molded in both polystyrene and polyethylene terephthalate and used to measure spheroids grown by InSphero and transferred into the plate following the protocol in Example 4. As shown in FIGS. 63 and 64, both polystyrene and polyethylene terephthalate plates enable the successful OCR measurement of spheroids; however, the measured signal is 1.5× higher for polyethylene terephthalate plates (FIG. 64) compared to the polystyrene plates (FIG. 63).

### Example 6

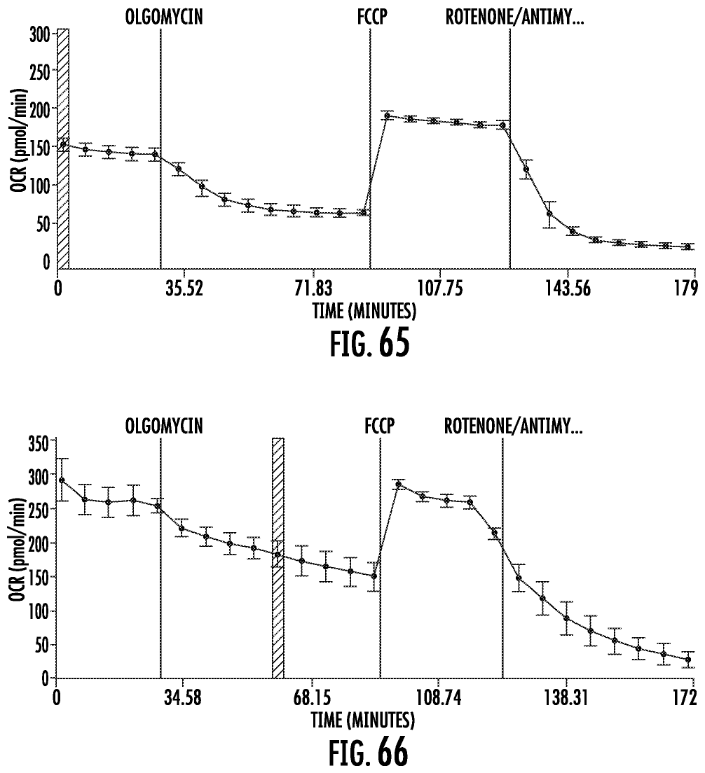

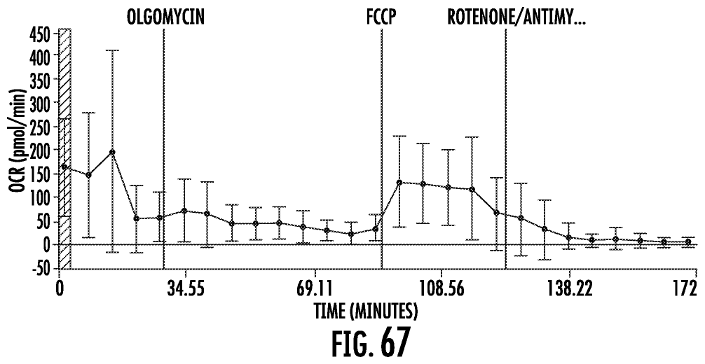

In order to show the advantages of growing and measuring spheroids in the same plate, we performed a side-by-side comparison of spheroids grown directly in the plate described here and spheroids transferred from a commercially available spheroid plate. In both cases, spheroids were grown following the same protocol and initial seeding of Panc1 cells. FIGS. 65-67 show the results of the Seahorse assay and includes a comparison of OCR signal for PANC1 spheroids grown in the plate disclosed in this invention versus spheroids grown on a commercial plate and transferred to the plate disclosed in this invention with and without centrifugation for measurement with a Seahorse Mito Stress Test. FIG. 65 shows the results when the spheroids are grown on the plate. FIG. 66 shows the results when the spheroids are transferred to the plate and centrifuged before the assay. FIG. 67 shows the results when spheroids are transferred and not centrifuged before the assay. The well-to-well variability is reduced when the centrifugation step is added. Spheroids that are grown in the plate showed more consistent results with no damaged spheroids observed.

Collectively, Applicant has demonstrated a plate configuration suitable for generating spheroids that remain centralized in the well thereby enabling metabolic measurement of the spheroid to be made. The plate design is also suitable for observing the centralized spheroids by an imaging device.

These and other modifications and variations to the present disclosure may be practiced by those of ordinary skill in the art, without departing from the spirit and scope of the present disclosure, which is more particularly set forth in the appended claims. In addition, it should be understood that aspects of the various embodiments may be interchanged both in whole or in part. Furthermore, those of ordinary skill in the art will appreciate that the foregoing description is by way of example only, and is not intended to limit the disclosure so further described in such appended claims.

1. An apparatus for containing a three-dimensional cellular material surrounded by a medium, the apparatus comprising a well having an open proximal end and a closed distal end that defines a bottom of the well, wherein the well defines a compartment having an interior surface and a sample nesting site for containing the three-dimensional cellular material surrounded by the medium, wherein a central indentation is located at the closed distal end of the well, a first concentric lip is located above the central indentation in a y-direction towards the open proximal end of the well, and a second concentric lip is located above the first concentric lip in the y-direction towards the open proximal end of the well, wherein the first concentric lip and the second concentric lip define a groove therebetween.

2. The apparatus of claim 1, wherein the first concentric lip has a first concave radius of curvature, the second concentric lip has a second concave radius of curvature, and the central indentation has a third concave radius of curvature.

3. The apparatus of claim 1, wherein the interior surface of the compartment near the closed distal end of the well is defined by a first convex radius of curvature between the central indentation and the first concentric lip and a second convex radius of curvature between the first concentric lip and the second concentric lip.

4. The apparatus of claim 1, wherein the bottom of the well is transparent, and/or wherein the interior surface comprises a polyethylene terephthalate, a polystyrene, a polypropylene, a polyvinyl chloride, a cyclic olefin copolymer, a polycarbonate, or a combination thereof.

5. The apparatus of claim 1, wherein a coating is deposited on at least a portion of the interior surface of the compartment, wherein the coating facilitates collection of the three-dimensional cellular material at the central indentation at the closed distal end of the well.

6. The apparatus of claim 1, wherein at least one protrusion is located at the closed distal end of the well, wherein the at least one protrusion is spaced radially from the central indentation towards a sidewall of the well.

7. (canceled)

8. The apparatus of claim 1, further comprising a probe that forms a seal at the closed distal end of the well when introduced into the compartment, wherein the first concentric lip or the second concentric lip defines a planar surface for receiving the probe.

9. The apparatus of claim 8, wherein the probe comprises a sensor for measuring a parameter, and/or wherein the well holds a volume of medium beneath the seal that is less than 200 microliters.

10-11. (canceled)

12. The apparatus of claim 1, wherein the three-dimensional cellular material comprises a spheroid, an organoid, or a tissue sample.

13. The apparatus of claim 1, wherein the apparatus comprises a plurality of wells defining a plurality of compartments.

14. A method for forming a three-dimensional cellular material, the method comprising the steps of:
a) providing a plate having at least one well having an open proximal end and a closed distal end that defines a bottom of the well, wherein the well defines a compartment having an interior surface and a sample nesting site for containing the three-dimensional cellular material, wherein a central indentation is located at the closed distal end of the well, a first concentric lip is located above the central indentation in a y-direction towards the open proximal end of the well, and a second concentric lip is located above the first concentric lip in the y-direction towards the open proximal end of the well, wherein the first concentric lip and the second concentric lip define a groove therebetween;
b) adding cells and a medium to the compartment; and
c) allowing the three-dimensional cellular material to form from the cells.

15. The method of claim 14, wherein the first concentric lip has a first concave radius of curvature, the second concentric lip has a second concave radius of curvature, and the central indentation has a third concave radius of curvature.

16. The method of claim 14, wherein the interior surface of the compartment near the closed distal end of the well is defined by a first convex radius of curvature between the central indentation and the first concentric lip and a second convex radius of curvature between the first concentric lip and the second concentric lip.

17. The method of claim 14, wherein the bottom of the well is transparent, and/or wherein the interior surface comprises a polyethylene terephthalate, a polystyrene, a polypropylene, a polyvinyl chloride, a polycarbonate, a cyclic olefin copolymer, or a combination thereof.

18. The method of claim 14, wherein a coating is deposited on at least a portion of the interior surface of the compartment, wherein the coating decreases a level of attachment of the three-dimensional material and/or other cellular material to the interior surface.

19. The method of claim 14, wherein at least one protrusion is located at the closed distal end of the well, wherein the at least one protrusion is spaced radially from the central indentation towards a sidewall of the well.

20. (canceled)

21. The method of claim 14, further comprising measuring a parameter of the three-dimensional cellular material by introducing a probe into the compartment to form a seal near the closed distal end of the well, wherein the first concentric lip or the second concentric lip defines a planar surface for receiving the probe.

22. The method of claim 21, wherein the probe comprises a sensor for measuring the parameter, and/or wherein the well holds a volume of medium beneath the seal that is less than 200 microliters.

23-24. (canceled)

25. The method of claim 14, wherein the three-dimensional cellular material comprises a spheroid, an organoid, or a tissue sample.

26. The method of claim 14, wherein the three-dimensional cellular material has a radius in a x-direction and a radius in a y-direction, wherein a ratio of the radius in the x-direction to the radius in the y-direction ranges from about 0.75 to about 1.25 after step (c).

27-29. (canceled)
